SEQUENCE LISTING (11 sequences)

SEQ ID NO: 1 - protein, 77 aa, Homo sapiens
RSVRAQRHTAMPKTQKYQPPSTNKNTKSQRRKGWPKTHPGGQQKQGTEASLQIRGKKKEQRREIGSRNAECRGKKGK

SEQ ID NO: 2 - protein, 74 aa, Oncorhynchus mykiss
RSVRAQRHTDMPRTPKVSTAVQNVDRGTERRTAGHPDKTKTKKKPLSGHSHPSCKEVHQKNSSRGNTGGRNTRM

SEQ ID NO: 3 - protein, 62 aa, Oncorhynchus mykiss
RSVRAQRHTDMPRTPKVSTAVQSVDRGTERRTAGHPDKTKPKKEVHQKNSSRGNTGGRNTRM

SEQ ID NO: 4 - protein, 47 aa, Oncorhynchus mykiss
RSVRAQRHTDMPRTPKKPLSGHSHPSCKEVHQKNSSRGNTGGRNTRM

SEQ ID NO: 5 - protein, 35 aa, Oncorhynchus mykiss
RSVRAQRHTDMPRTPKEVHQKNSSRGNTGGRNYRM

SEQ ID NO: 6 - DNA, 18 bp, Artificial
  Synthetic Primer
cttgtggccgtttacgtc

SEQ ID NO: 7 - DNA, 18 bp, Artificial
  Synthetic Primer
gcacagcacccagacaag

SEQ ID NO: 8 - protein, 74 aa, Rattus norvegicus
  MISC_FEATURE (1)..(74): rat Ea4 peptide
RSVRAQRHTDMPRTPKVSTAVQNVDRGTERRTAQHPDKTKTKKKPLSGHSHPSCKEVHQKNSSRGNTGGRNYRM

SEQ ID NO: 9 - protein, 60 aa, Rattus norvegicus
  MISC_FEATURE (1)..(60): rat Ea3 peptide
RSVRAQRHTDMPRTPKVSTAVQSVDRGTERRTAQHPDKPKKEVHQKNSSRGNTGGRNYRM

SEQ ID NO: 10 - protein, 47 aa, Rattus norvegicus
  MISC_FEATURE (1)..(47): rat Ea2 peptide
RSVRAQRHTDMPRTPKKPLSGHSHPSCKEVHQKNSSRGNTGGRNYRM

SEQ ID NO: 11 - protein, 35 aa, Rattus norvegicus; identical to SEQ ID NO: 5
  MISC_FEATURE (1)..(35): rat Ea1 peptide

(19) United States
(12) Patent Application Publication
(10) Pub. No.: US 2009/0221072 A1
(43) Pub. Date: Sep. 3, 2009

(54) COMPOSITIONS AND METHODS FOR MODULATING CELL DIFFERENTIATION

(76) Inventors: Thomas T. Chen, Storrs, CT (US); Maria J.M. Chen, Storrs, CT (US)

Correspondence Address: MCCARTER & ENGLISH, LLP HARTFORD;CITYPLACE I, 185 ASYLUM STREET, HARTFORD, CT, 06103, US

(21) Appl. No.: 12/465,053

(22) Filed: May 13, 2009

Related U.S. Application Data
(63) Continuation-in-part of application No. 11/354,484, filed on Feb. 15, 2006.
Application No. 11/354,484 is a continuation-in-part of application No. 11/799,623, filed on May 2, 2007.
(60) Provisional application No. 61/052,779, filed on May 13, 2008.
(60) Provisional application No. 61/052,781, filed on May 13, 2008.

(51) Int. Cl. C12N 5/06

(52) U.S. Cl. 435/377; 435/325

(57) ABSTRACT

Compositions and methods are described for using the Ea4-peptide of pro-IGF-I or human Eb-peptide of pro-IGF-I to inhibit hematopoiesis and to induce differentiation of neuroblastoma cells and neuronal stem cells.

### CROSS-REFERENCE TO RELATED APPLICATIONS

Under 35 U.S.C. § 119(e) this application claims the benefit to U.S. Provisional Patent Applications 61/052,779 and 61/052,781 filed: May 13, 2008; and is a Continuation-in-Part of U.S. patent application Ser. No. 11/354,484 filed Feb. 15, 2006, entitled: Compositions and methods for inducing apoptosis in tumor cells; and U.S. patent application Ser. No. 11/799,623 filed May 2, 2007, entitled: Anti-tumor activity of Ea-4-peptide of pro-IGF-I; the disclosures of which are hereby incorporated by reference in their entirety.

### SEQUENCE LISTING

The present application hereby incorporates by reference, in its entirety, the Sequence Listing submitted herewith. An electronic version of the Sequence Listing is being filed herewith, file name: 97511_{—}00010_ST25.txt , size: 7 KB, created: May 13, 2009 using PatentIn 3.4 software on Windows XP; the contents of which are identical to the written version of the Sequence Listing.

### FIELD OF THE INVENTION

The present invention relates generally to therapeutic uses of IGF-I peptides. In particular, the present invention relates to use of IGF-I, E-domain peptides for modulating the differentiation of primary and/or progenitor cells.

### BACKGROUND

Research on progenitor cells, for example, stem cells, is advancing knowledge about how an organism develops from a single cell and how healthy cells replace damaged cells in adult organisms. This promising area of science is also leading scientists to investigate the possibility of using cell-based therapies to treat disease, which is often referred to as regenerative or reparative medicine (i.e., a treatment in which stem cells are induced to differentiate into the specific cell type required to repair damaged or destroyed cell populations or tissues).

Stem cells have two important characteristics that distinguish them from other types of cells. First, they are unspecialized cells that renew themselves for long periods through cell division. The second is that under certain physiologic or experimental conditions, they can be induced to become cells with special functions such as the beating cells of the heart muscle, specific blood cell types, neurons or the insulin-producing cells of the pancreas.

Currently, determining the mechanisms that underlie adult stem cell plasticity is an active area of research. If such mechanisms can be identified and controlled, existing stem cells and even primary cells taken from a healthy tissue might be induced to repopulate and repair a diseased tissue. One of the main challenges and current needs in primary and stem cell research, and consequently in the development of cell-based therapies, are compositions and methods for delaying or suspending the differentiation of primary or progenitor cells so that they can be propagated and cultured in vitro or ex vivo. The cultured cells can then be induced to differentiate into the desired cell type, either in vitro or in vivo.

The mature form of IGF-I is a basic protein of 7.5-kDa. The pre-pro-peptides of the IGF-I consist of an amino-terminal signal peptide, followed by the mature peptide with B, C, A and D domains, and a carboxyl-terminal E domain (See FIG. 1A for a schematic representation). The signal peptide at the amino-terminal end and the E-domain peptide at the carboxy-terminal end of the pre-pro-peptide are proteolytically cleaved from the peptide to result in the mature, biochemically active species. Tian et al. (1999) have reported that recombinant rainbow trout Ea-2-, Ea-3- and Ea-4-peptides possess mitogenic activity in several non-transformed cell lines, including NIH 3T3 cells and caprine mammary epithelium cells (CMEC) (Panschenko et al., 1997). Trout Ea-2-and Ea-4-peptide contains a signal motif for peptidyl C-terminal amidation (Shamblott and Chen, 1993; Barr, 1991), and a bipartite consensus nuclear localization sequence is also present in Ea-4-peptide (Shamblott and Chen, 1993; Dingwall and Laskey, 1991).

The present inventors have now discovered that the E-peptides possess novel biological activities including the modulation of primary and progenitor cell differentiation. The modulation of primary and progenitor cell differentiation allows for the creation of cell lines useful for performing cell-based therapies, producing or screening therapeutic biomolecules, and are useful as a tool for further research into the genes/proteins/signaling pathways that mediate differentiation into specific cell types.

### SUMMARY

Described herein are compositions and methods for modulating cell differentiation. The present invention is based on the surprising and unexpected discovery that Insulin-like Growth Factor-1 (IGF-1) E-domain peptides (E-peptides) can modulate the differentiation of primary and progenitor cells. For example, E-peptides can inhibit hematopoietic development, and promote differentiation of neuronal primary cells, progenitor cells, and/or neuronal stem cells (herein, collectively, “progenitor cells”), for example, primary pituitary cells, pituitary stem cells, and neuroblastoma cells.

Therefore, in one aspect, the invention relates to compositions and methods for inhibiting the differentiation of a progenitor cell, for example, a stem cell such as a hematopoietic stem cell, comprising treating a progenitor cell with a composition comprising an effective amount of an insulin-like growth factor E-domain peptide (E-peptide) together with at least one of a pharmaceutically acceptable carrier, excipient or adjuvant, wherein the composition inhibits cell differentiation.

In another aspect, the invention relates to compositions and methods for promoting the differentiation of a neuronal progenitor cell, neuronal stem cell, and/or a neuronal tumor progenitor cell, for example a neuroblastoma cell, comprising treating a neuronal progenitor cell with a composition comprising an effective amount of an insulin-like growth factor E-domain peptide together with at least one of a pharmaceutically acceptable carrier, excipient or adjuvant, wherein the composition promotes cell differentiation.

In another aspect, the invention relates to an immortalized and/or undifferentiated progenitor cell, for example, a hematopoietic progenitor cell, that has been generated using a method of the invention.

In another aspect, the invention relates to a differentiated neuronal progenitor cell, for example, a primary neuronal cell or neuroblastoma cell, that has been generated according to a method of the invention.

In another aspect, the invention relates to an undifferentiated hematopoietic progenitor cell, for example, a hematopoietic stem cell that has been generated according to a method of the invention.

In another aspect, the invention relates to nucleic acid constructs (i.e., plasmids, vectors, expression constructs, and the like) comprising a nucleic acid encoding for an E-domain polypeptide or fragment thereof, operably linked with at least one DNA regulatory element, for example, a transcription, and/or replication regulatory element.

In certain aspects or embodiments described herein, the composition comprises at least one E-domain peptide having an amino acid sequence with at least about 30%, 40%, 50%, 60%, 70%, 80%, or 90% sequence homology with an hEb-domain peptide having the amino acid sequence of SEQ ID NO:1.

The preceding general areas of utility are given by way of example only and are not intended to be limiting on the scope of the present disclosure and appended claims. Additional objects and advantages of the present invention will be appreciated by one of ordinary skill in the art in light of the instant claims, description, and examples. For example, the various aspects and embodiments of the invention may be utilized in numerous combinations, all of which are expressly contemplated by the present description. These additional objects and advantages are expressly included within the scope of the present invention.

### BRIEF DESCRIPTION OF THE DRAWINGS

The accompanying drawings, which are incorporated into and form a part of the specification, illustrate several aspects of the present invention and, together with the description, serve to explain the principles of the invention. The drawings are only for the purpose of illustrating embodiments of the invention and are not to be construed as limiting the invention.

FIG. 1 (A) is a schematic representation of the subforms of mammalian and rainbow trout pro-IGF-1 pro-peptides. B, C, A, D, and E indicate different domains of the IGF-1 peptides. (B) shows the amino sequence alignment of hEb (SEQ ID NO: 1), rtEa-4 (SEQ ID NO:2), rtEa-3 (SEQ ID NO:3), rtEa-2 (SEQ ID NO: 4), and rtEa-1 (SEQ ID NO: 5).

FIG. 2 is a schematic representation of the gene constructs used in the transfection of target cells. IGF-I-sp: signal peptide of hlGF-I; Ea-4 cDNA: cDNA of rtEa-4-peptide; hEb cDNA: cDNA of the human Eb peptide; EGFP: coding region of the enhanced green fluorescence protein gene; IRES: internal ribosome entry site.

FIG. 3. Hemoglobinized red blood cells were captured by diaminofluorine (DAF) staining at 28 hours post fertilization (hpf) and 35 hpf. Zebrafish embryo was dechorinated at 28 hpf and 35 hpf. After DAF staining for 20 min as described in materials and methods, five images of each embryo were taken under a microscope and Z-stacked using the ImageJ software (http://rsb.info.nih.gov/ij) to generate multi-focus image. The deep blue coloration shows the hemoglobinized red blood cells in embryo (arrows).

FIG. 4. Effect of rtEa4-peptide on red blood cell development in zebrafish embryos. Zebrafish embryos were injected with rtEa4-peptide (1 pmol) at 2.5 hpf, and the animals were stained with DAF to visualize the red blood cells (i.e., DAF staining for detection of pseudo-peroxidase activity of normal and defective embryos). A. normal embryo, B. defective embryo received recombinant rtEa4-peptide (1 pmol) at 2.5 hpf. i, head and yolk sac; ii, body; iii, tail. Arrows indicate stained dark blue hemoglobin. The results show that very few red blood cells are observed in embryos treated with rtEa4-peptide, suggesting that rtEa4-peptide inhibits the development of red blood cells.

FIG. 5. Temporal expression profile of fli1a, c-myb, mpx, l-plastin, and ikaros gene in embryonic development of zebrafish.

FIG. 6. Effect of rtEa4-peptide on morphological differentiation of human neuroblastoma cells (SK-N-F1 and IMR32). SK-N-F1 and IMR32 cells were cultured in DMEM/F12 (1:1) supplemented with 10% FBS and 10 μg/ml of rtEa4-peptide. Cell morphology was observed under an inverted microscope (1×50 Olympus). Bar indicates 50 μm.

FIG. 7. Single-cell clones of pituitary cell lines. Primary cells of rainbow trout pituitary were cultured in a CO₂-independent medium supplemented with 10% FBS and 40 μg/ml rtEa4-peptide. Single-cell clones were isolated and sub-cultured for 120 passages. The cells were seeded in low density (a) and high density (b) and observed under a microscope (1×50 Olympus). Bar indicates 100 μm.

FIG. 8. Immunostaining of growth hormone (GH) and prolactin (PRL) in trout pituitary single-cell clones. Single-cell clone of trout pituitary cells lines were plated on cover slips, fixed, and stained with antisera of trout GH and PRL, and counter stained with goat anti-rabbit IGG-FITC conjugate. The results showed that pituitary single-clone cells produce GH and PRL. NS=nonspecific antiserum; GH Ab=anti-GH serum; PRL Ab=anti-PRL serum.

### DETAILED DESCRIPTION

Described herein are compositions and methods for modulating progenitor cell differentiation. The present invention is based on the surprising and unexpected discovery that Insulin-like Growth Factor-1 (IGF-1) E-domain peptides (or E-peptides) can inhibit hematopoiesis, yet promote the differentiation of neuronal stem cells, in particular, primary pituitary stem cells and tumor progenitor cells, in particular, neuroblastoma cells. This biological activity, which could not have been predicted, a priori, indicates that E-peptides are useful for the creation of cell lines useful for performing cell-based therapies, producing or screening therapeutic biomolecules, and are useful tools for further research into the genes/proteins/pathways that mediate differentiation into specific cell types.

The following U.S. patent applications and U.S. patents discuss subject matter related to the present invention and are incorporated herein by reference: U.S. patent application Ser. No. 11/354,484 filed Feb. 15, 2006; U.S. patent application Ser. No. 11/799,623 filed May 2, 2007; and U.S. Pat. Nos. 6,358,916; 6,610,302; 7,118,752; and 7,250,169.

Insulin-like growth factors (IGF's) are mitogenic peptides that regulate embryonic development, post-natal growth and cellular differentiation in vertebrates. The functions of mature IGF peptides have been extensively studied in various in vitro and in vivo systems. IGF's, including IGF-I and IGF-II, are among the members of a family of structurally and evolutionarily related peptides that also include insulin and relaxins. Like many hormones, IGF's are initially translated as pre-pro-peptides that undergo post-translational processing to result in the mature peptides.

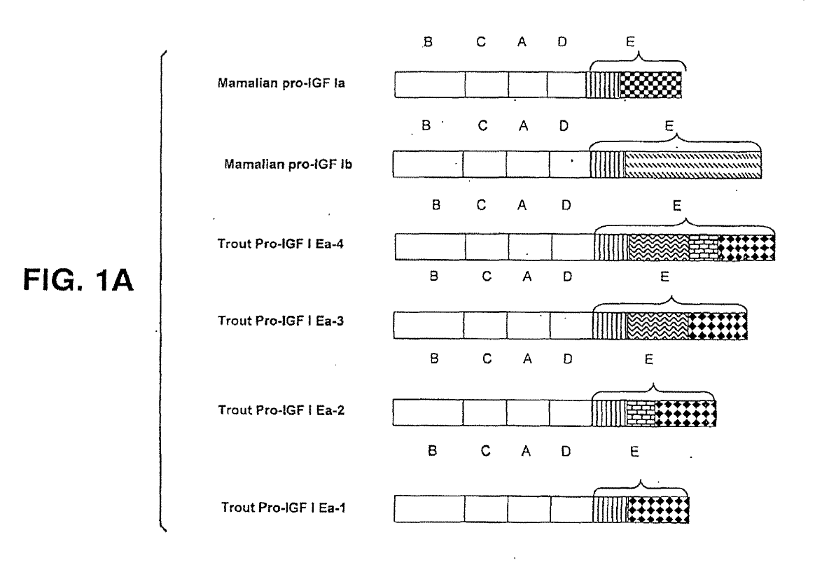

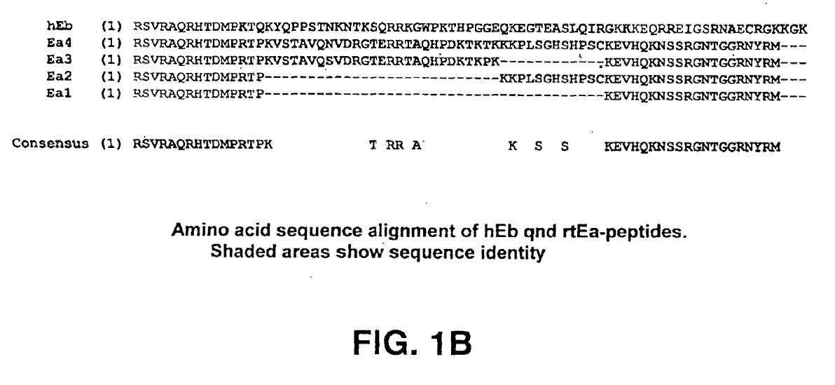

The mature form of IGF-I is a basic protein of 7.5 kDa. The pre-pro-peptides of the IGF-I consist of an amino-terminal signal peptide, followed by the mature peptide with B, C, A and D domains, and a carboxyl-terminal E domain (See FIG. 1A). The signal peptide at the amino-terminal end and the E-domain peptide at the carboxy-terminal end of the pre-pro-peptide are proteolytically cleaved from the peptide to result in the mature, biochemically active species.

To date, multiple forms of pro-IGF-I have been identified in species from fish to mammals (Shamblott, Chen, Mol Mar Biol Biotechnol. 2: 351-61, 1993; Rotwein, Proc. Natl. Acad. Sci USA, 83:77-81, 1986). See, for example, accession numbers: P16501 (Xenopus laevis), P05017 (Mus musculus), Q95222 (Oryctolagus cuniculus), P08025 (Rattus norvegicus), Q90325 (Cyprinus carpio), CAA40092 (Homo sapiens), NP_{—}001071296 (Bos taurus), P10763 (Ovis aries); P16545 (Sus scrofa); Q02815 (Oncorhynchus mykiss); P17085 (Oncorhynchus kisutch); P18254 (Gallus gallus); P51458 (Equus caballus); NP_{—}571900 (Danio rerio); and P33712 (Canis familiaris); which are incorporated herein by reference.

In humans, three alternative spliced isoforms of pro-IGF-I (pro-IGF-I-a pro-IGF-I-b and pro-IGF-I-c) have been reported (Rotwein, Proc. Natl. Acad. Sci USA, 1986; Rotwein, et al., J. Biol. Chem., 261: 4828-32, 1986; Chew, et al., Endocrinology, 136: 1939-44, 1995). These three pro-IGF-I isoforms differ only in the carboxyl-terminal E-domain regions that are normally removed in vivo from the mature IGF-I. The E-domains of pro-IGF-I-a, pro-IGF-I-b and pro-IGF-I-c contain 35, 77 and 40 amino acid residues, respectively. The first 15 amino acid residues at the N-terminus of E-domains (referred to as the common region) share identical sequences. The amino acid sequences following the common region vary between the three isoforms of human pro-IGF-I (see FIG. 1B).

Similar diversity of pro-IGF-I E-domains is also found in rainbow trout (Oncorhynchus mykis), where four different isoforms have been identified, designated for consistent reference herein as pro-IGF-I Ea-1, Ea-2, Ea-3 and Ea-4 (Shamblott, Chen, Mol. Mar. Biol. Biotech., 1993). Nucleotide sequence comparison of the four size forms of rainbow trout IGF-I mRNAs is consistent with the above observations concerning the Ea peptides in that the size differences among these mRNA species are due to insertions or deletions in the E domain regions of the molecules (See FIGS. 1A and 1B). The predicted amino acid residues of the common region of the four Ea peptides share identical sequences among themselves, as well as with pro-IGF-I E-peptides of human, mouse, and rat species (See FIG. 1B). The presence of the C-terminal 20 amino acid residues, sharing 70% identity with their human counterparts, identifies them as a-type E-peptides. The Ea-I peptide of the rainbow trout (rt) pro-IGF-I (SEQ ID NO: 5) is a polypeptide of 35 amino acid residues, comprising the first 15 and the last 20 amino acid residues. Ea-2 (SEQ ID NO: 4) and Ea-3 (SEQ ID NO: 3) peptides differ from Ea-1 by virtue of either a 12- or 27-amino acid residue insertion between the first and last segments of the Ea-1-peptide sequence, respectively (see FIG. 1B). The Ea-4 peptide (SEQ ID NO: 2) contains both insertions. The predicted numbers of amino acid residues in each E-peptide are, thus, 35 (SEQ ID NO: 5), 47 (SEQ ID NO: 4), 62 (SEQ ID NO: 3) and 74 (SEQ ID NO: 2), respectively. There has not been any report on the presence of b-type IGF-I mRNA in rainbow trout (Shamblott and Chen, 1993).

FIG. 1B shows the amino acid sequences of the human Eb peptide (hEb) (SEQ ID NO:1) and the trout Ea peptides. Despite not having complete homology at the primary level, studies indicate that hEb and trout Ea-4 peptide have very similar tertiary structures, particularly in the amino-terminal region containing the common sequences, and can compete effectively for binding to cell receptors specific to E-domain peptides.

Despite the presence of multiple E-domain variants, assigning biological function to the IGF E-domains has been elusive. Proteolytic processing of the pro-IGF's, resulting in the cleavage of E-domains from IGF's, is believed to be similar to the cleavage of the C-peptide of proinsulin (Foyt, et al., Insulin-Like Growth Factors: Molecular and Cellular Aspects, pp1-16. Boca Raton: CRC press, 1991). In the past, it was generally accepted that E-domains, like the C-peptide of pro-insulin, possess little or no biological activity other than their potential roles in the biosynthesis of mature IGF. The C-peptide of pro-insulin is believed to have an essential function in the biosynthesis of insulin in linking the A and B chains in a manner that allows correct folding and inter-chain disulfide bond formation. In spite of the earlier reports indicating certain physiological effects of the insulin C-peptide (Johansson, et al., Diabetologia, 35: 121-28, 1992; Johansson, et al., Diabetologia, 35: 1151-58, 1992; Johansson, et al., J. Clin. Endo. Metab., 77: 976-81, 1993), it has not been widely accepted until recently.

The C-peptide has now been shown to have many beneficial effects on various abnormalities in diabetic animal models and patients (Ido, et al., Science, 277: 563-66, 1997; Forst, et al., J. Clin. Invest. 101: 2036-41, 1998; Sjoquist, et al., Kidney Int., 54: 758-64, 1998). Moreover, recent studies further demonstrated specific binding of C-peptide to cell surfaces in a manner that suggests the presence of G-protein-coupled membrane receptors (Rigler, et al., Proc. Natl. Acad. Sci USA, 96: 13318-23, 1999). It is now thought that C-peptide may thereby stimulate specific intracellular signal transduction leading to the biological activities of C-peptide (Wahren, et al., Am. J. Physiol. Endo. Metab. 278: E759-68, 2000; Kitamura, et al., Biochem J., 355: 123-29, 2001).

Recombinant Ea-2, Ea-3 and Ea-4 peptides of rainbow trout pro-IGF-I possess mitogenic activity in cultured BALB/3T3 fibroblast (Tian, et al., Endocrinology, 140: 3387-90, 1999). In addition to mitogenic activity, trout pro-IGF-I Ea-2 and Ea-4 peptides possess activities including induction of morphological change, enhancement of cell attachment, restoration of anchorage-dependent cell division behavior, and reduction of the invasiveness of aggressive cancer cells. Since similar morphological change has also been induced in a hepatoma cell line of Peoceliposis lucida (desert guppy) by treatment with the trout Ea-4 peptide, this observation rules out the possibility that the effects of trout pro-IGF-I Ea-4-peptide on human cancer cells are the consequence of artifact. Presently, the inventors have identified previously unknown biological effects of E-peptides on progenitor cells, which have significant commercial and therapeutic implications.

Hematopoiesis (or haematopoiesis) refers to the formation and development of the cells of the blood. All of the cellular components of the blood are derived from hematopoietic progenitor or stem cells. The term multipotent or pluripotent refers to the ability of a cell to become several different types of cell (but not all types in a germ layer, i.e., omnipotent). As a stem cell matures it undergoes changes in gene expression (the rate at which a gene is converted to its encoded products) that limit the cell types that it can become and move it closer to a specific cell type. These changes can often be tracked by monitoring the presence of proteins on the surface of the cell. Each successive change moves the cell closer to its final choice of cell type and further limits its potential cell type until it is fully differentiated. Current research suggests that is the location of blood cells that makes the cell determination and not vice versa. For instance, the thymus provides an environment for thymocytes to differentiate into a variety of different functional T cells.

In humans, hematopoiesis begins in the yolk sac in the first weeks of embryonic development. By the third month of gestation, stem cells migrate to the fetal liver and then to the spleen (between 3-7 months gestation these two organs play a major hempatopoietic role). Next, the bone marrow becomes the major hematopoietic organ and hematopoiesis ceases in the liver and spleen. Every functional specialized mature blood cell is derived from a common stem cell. Therefore, these stem cells are considered, pluripotent.

All blood cells develop from pluripotent stem cells that are found in the red bone marrow. Stem cells make up 10% of cord blood cells and <1% of all adult blood cells. Stem cells are able to proliferate as well as differentiate into the different types of blood cells. They are also able to renew themselves.

It has been estimated that there is approximately 1 stem cell per 10⁴ bone marrow cells. These stem cells represent a self-renewing population of cells. These cells also must have the potential to differentiate and to become committed to a particular blood cell lineage. Due to the low frequency of these cells and the well-known difficulties in attempting to culture these cells in vitro, stem cells have been very difficult to study. However, in vivo studies in mice have shown with lethal irradiation (950 rads) death occurs within 10 days. If a mouse is infused with only 10⁴-10⁵ bone marrow cells from a syngeneic donor, the hematopoietic system can be completely restored. Therefore, there must be at least one stem cell in a population of bone marrow cells of this size. In theory, a single stem cell is capable of completely restoring the hematopoietic process.

Initial differentiation of pluripotent stem cells will be along one of two major pathways (lymphoid or myeloid). These, “multipotent” stem cells then become progenitor cells for each type of mature blood cell. These cells have lost the capacity for self-renewal and are committed to a given cell lineage, for example, T and B cell progenitors, and progenitor cells for erythrocytes, neutrophils, eosinophils, basophils, monocytes, mast cells, and platelets. (Table 1).

TABLE 1
Hematopoietic Stem Cell Progenitors

| | | | | neutrophil, monocyte, | |
|---|---|---|---|---|---|
| | | | | macrophage, eosinophil, | |
| | Myeloid | progenitor cells for each | | erythrocyte, megakaryocytes, | |
| | Stem Cell | cell type | | mast cells, basophils | |
| Multipotent | Lymphoid | progenitor B | precursor B | mature B | Plasma Cell |
| hematopoietic | Stem Cell | | | lymphocyte | Memory B |
| stem cell | | | | | Cell |
| | | progenitor T | precursor Tc | mature Tc | CTL memory |
| | | | | | Tc |
| | | | precursor | mature Th | Th1 |
| | | | Th | | Th2 |

Progenitor commitment depends upon the acquisition of responsiveness to certain growth factors. The particular microenvironment within which the progenitor cell resides controls differentiation. The hematopoietic cells grow and mature on a meshwork of stromal cells, which are nonhematopoietic cells that support the growth and differentiation of the hematopoietic cells. These stromal cell types include: fat cells, endothelial cells, fibroblasts, and macrophages. These cells provide a hematopoietic-inducing microenvironment. The microenvironment consists of the actual cellular matrix and either membrane-bound or diffusable growth factors, for example, Colony Stimulating Factors, multilineage colony-stimulating factor (multi-CSF or IL-3), granulocyte-macrophage colony stimulating factor (GM-CSF), macrophage colony stimulating factor (M-CSF), granulocyte colony-stimulating factor (G-CSF), Erythropoietin—Induces terminal erythrocyte development and regulates RBC production, and ILs 4-9. These growth factors are present at extremely low concentrations and biological activity at concentrations as low as 10⁻¹² M.

Commitment of a progenitor cell is associated with the expression on the cell membrane of membrane receptors that are specific for particular cytokines. Hematopoiesis is a continuous process throughout adulthood. In a healthy adult, production of mature blood cells equals their loss. This process is regulated by complex mechanisms. Cell division and differentiation during hematopoiesis are balanced by apoptosis. It has been estimated that the average human must produce 3.7×10¹¹ blood cells per day. If the apoptosis mechanism fails, a leukemic state can occur.

Fetal Hematopoiesis. The first blood cells formed are erythrocytes or red blood cells (RBCs). At 2 to 8 weeks primitive nucleated erythroid cells are found in the yolk sac; they contain hemoglobin but don't mature to fully developed RBCs; formation occurs in aggregates of blood cells in the yolk sac, called blood islands. During the 2nd month extramedullary hematopoiesis develops; yolk sac cells migrate to the liver. Granulocytes also appear in the liver during the 2nd month and all adult organs are recognizable. The spleen also contributes to hematopoiesis at this point. During the 4th month medullary hematopoiesis develops when the bone marrow begins to contribute to hematopoiesis. During the 5th month bone marrow takes over as chief production site and continues throughout life.

Postnatal Hematopoiesis. At birth the liver and spleen have ceased production of blood cells and hematopoiesis is occurring in the red bone marrow of almost every bone (axial and appendicular skeletons). As a child develops and matures (beginning at 4 years) the hematopoietic activity begins to move to the axial skeleton (flat bones, skull, ribs, sternum, clavicle, vertebrae, pelvic bones) and proximal ends of long bones (humerus and femur). This move is completed by age 18.

Hematopoiesis in Adults. Remaining marrow cavities are replaced with fat (yellow bone marrow). By age 40 the marrow in sternum, ribs, pelvis and vertebrae is composed of equal amounts of hematopoietic tissue and fat. In times of great demand the marrow in the long bone shafts may become hematopoietic again. Extramedullary hematopoiesis may occur under two conditions: If the bone marrow is no longer functional; When the bone marrow is not able to keep up with the demand for blood cells. When extramedullary hematopoiesis occurs, the liver and spleen will become enlarged.

Stem Cells.

Stem cells can give rise to specialized cells. When unspecialized stem cells give rise to specialized cells, the process is called differentiation. Scientists are just beginning to understand the signals inside and outside cells that trigger stem cell differentiation. The internal signals are controlled by a cell's genes, which are interspersed across long strands of DNA, and carry coded instructions for all the structures and functions of a cell. The external signals for cell differentiation include chemicals secreted by other cells, physical contact with neighboring cells, and certain molecules in the microenvironment. Questions about stem cell differentiation remain. For example, are the internal and external signals for cell differentiation similar for all kinds of stem cells? Can specific sets of signals be identified that promote differentiation into specific cell types? Addressing these questions is critical because the answers may lead scientists to find new ways of controlling stem cell differentiation in the laboratory, thereby growing cells or tissues that can be used for specific purposes including cell-based therapies. For additional detail on stem cells; See, Zhou J, Zhang Y., Cancer stem cells: models, mechanisms and implications for improved treatment. Cell Cycle. Mar. 19, 2008; 7(10); Einstein O, Ben-Hur T., The Changing Face of Neural Stem Cell Therapy in Neurologic Diseases. Arch Neurol. 2008 Apr; 65(4):452-456; Hamadani M, Awan F T, Copelan E A., Hematopoietic stem cell transplantation in adults with acute myeloid leukemia. Biol Blood Marrow Transplant. 2008 May; 14(5):556-67; Papayannopoulou T, Scadden D T., Stem-cell ecology and stem cells in motion. Blood. Apr. 15, 2008; 111(8):3923-30; which are hereby incorporated herein by reference.

Adult stem cells typically generate the cell types of the tissue in which they reside. A blood-forming adult stem cell in the bone marrow, for example, normally gives rise to the many types of blood cells such as red blood cells, white blood cells and platelets. Until recently, it had been thought that a hematopoietic stem cell could not give rise to the cells of a very different tissue, such as nerve cells in the brain. However, a number of experiments over the last several years have raised the possibility that stem cells from one tissue may be able to give rise to cell types of a completely different tissue, a phenomenon known as transdifferentiation or plasticity. Examples of such plasticity include blood cells becoming neurons, liver cells that can be made to produce insulin, and hematopoietic stem cells that can develop into heart muscle. Therefore, exploring the possibility of using adult stem cells for cell-based therapies has become a very active area of investigation by researchers.

An adult stem cell is an undifferentiated cell found among differentiated cells in a tissue or organ, can renew itself, and can differentiate to yield the major specialized cell types of the tissue or organ. The primary roles of adult stem cells in a living organism are to maintain and repair the tissue in which they are found. Some scientists now use the term somatic stem cell instead of adult stem cell. Unlike embryonic stem cells, which are defined by their origin (the inner cell mass of the blastocyst), the origin of adult stem cells in mature tissues is unclear.

Research on adult stem cells has recently generated a great deal of excitement. Scientists have found adult stem cells in many more tissues than they once thought possible. This finding has led scientists to ask whether adult stem cells could be used for transplants. In fact, adult blood forming stem cells from bone marrow have been used in transplants for 30 years. Certain kinds of adult stem cells seem to have the ability to differentiate into a number of different cell types, given the right conditions. If this differentiation of adult stem cells can be controlled in the laboratory, these cells may become the basis of therapies for many serious common diseases.

The history of research on adult stem cells began about 40 years ago. In the 1960s, researchers discovered that the bone marrow contains at least two kinds of stem cells. One population, called hematopoietic stem cells, forms all the types of blood cells in the body. A second population, called bone marrow stromal cells, was discovered a few years later. Stromal cells are a mixed cell population that generates bone, cartilage, fat, and fibrous connective tissue.

Also in the 1960s, scientists who were studying rats discovered two regions of the brain that contained dividing cells, which become nerve cells. Despite these reports, most scientists believed that new nerve cells could not be generated in the adult brain. It was not until the 1990s that scientists agreed that the adult brain does contain stem cells that are able to generate the brain's three major cell types—astrocytes and oligodendrocytes, which are non-neuronal cells, and neurons, or nerve cells.

Normal differentiation pathways of adult stem cells. In a living animal, adult stem cells can divide for a long period and can give rise to mature cell types that have characteristic shapes and specialized structures and functions of a particular tissue. The following are examples of differentiation pathways of adult stem cell: (i) Hematopoietic stem cells give rise to all the types of blood cells: red blood cells, B lymphocytes, T lymphocytes, natural killer cells, neutrophils, basophils, eosinophils, monocytes, macrophages, and platelets; (ii) Bone marrow stromal cells (mesenchymal stem cells) give rise to a variety of cell types: bone cells (osteocytes), cartilage cells (chondrocytes), fat cells (adipocytes), and other kinds of connective tissue cells such as those in tendons; (iii) neural stem cells in the brain give rise to its three major cell types: nerve cells (neurons) and two categories of non-neuronal cells—astrocytes and oligodendrocytes; (iv) Epithelial stem cells in the lining of the digestive tract occur in deep crypts and give rise to several cell types: absorptive cells, goblet cells, Paneth cells, and enteroendocrine cells; (v) Skin stem cells occur in the basal layer of the epidermis and at the base of hair follicles. The epidermal stem cells give rise to keratinocytes, which migrate to the surface of the skin and form a protective layer. The follicular stem cells can give rise to both the hair follicle and to the epidermis.

Adult stem cell plasticity and transdifferentiation. A number of experiments have suggested that certain adult stem cell types are pluripotent. This ability to differentiate into multiple cell types is called plasticity or transdifferentiation. The following list offers examples of adult stem cell plasticity that have been reported during the past few years: (i) Hematopoietic stem cells may differentiate into: three major types of brain cells (neurons, oligodendrocytes, and astrocytes); skeletal muscle cells; cardiac muscle cells; and liver cells; (ii) Bone marrow stromal cells may differentiate into: cardiac muscle cells and skeletal muscle cells; (iii) Brain stem cells may differentiate into: blood cells and skeletal muscle cells.

Human embryonic and adult stem cells each have advantages and disadvantages regarding potential use for cell-based regenerative therapies. Of course, adult and embryonic stem cells differ in the number and type of differentiated cells types they can become. Embryonic stem cells can become all cell types of the body because they are pluripotent. Adult stem cells are generally limited to differentiating into different cell types of their tissue of origin. However, some evidence suggests that adult stem cell plasticity may exist, increasing the number of cell types a given adult stem cell can become.

Large numbers of embryonic stem cells can be relatively easily grown in culture, while adult stem cells are rare in mature tissues and methods for expanding their numbers in cell culture have not yet been worked out. This is an important distinction, as large numbers of cells are needed for stem cell replacement therapies.

A potential advantage of using stem cells from an adult is that the patient's own cells could be expanded in culture and then reintroduced into the patient. The use of the patient's own adult stem cells would mean that the cells would not be rejected by the immune system. This represents a significant advantage as immune rejection is a difficult problem that can only be circumvented with immunosuppressive drugs. Embryonic stem cells from a donor introduced into a patient could cause transplant rejection. However, whether the recipient would reject donor embryonic stem cells has not been determined in human experiments.

Potential uses of stem cells. Studies of human embryonic stem cells may yield information about the complex events that occur during development. A primary goal of this work is to identify how undifferentiated stem cells become differentiated. Scientists know that turning genes on and off is central to this process. Some of the most serious medical conditions, such as cancer and birth defects, are due to abnormal cell division and differentiation. A better understanding of the genetic and molecular controls of these processes may yield information about how such diseases arise and suggest new strategies for therapy. A significant hurdle to this use and most uses of stem cells is that scientists do not yet fully understand the signals that turn specific genes on and off to influence the differentiation of the stem cell.

Stem cells could also be used to test new drugs. For example, new medications could be tested for safety on differentiated cells generated from human pluripotent cell lines. Other kinds of cell lines are already used in this way. Cancer cell lines, for example, are used to screen potential anti-tumor drugs. But, the availability of pluripotent stem cells would allow drug testing in a wider range of cell types. However, to screen drugs effectively, the conditions must be identical when comparing different drugs. Therefore, scientists will have to be able to precisely control the differentiation of stem cells into the specific cell type on which drugs will be tested. Current knowledge of the signals controlling differentiation fall well short of being able to mimic these conditions precisely to consistently have identical differentiated cells for each drug being tested.

Perhaps the most important potential application of human stem cells is the generation of cells and tissues that could be used for cell-based therapies. Today, donated organs and tissues are often used to replace ailing or destroyed tissue, but the need for transplantable tissues and organs far outweighs the available supply. Stem cells, directed to differentiate into specific cell types, offer the possibility of a renewable source of replacement cells and tissues to treat diseases including Parkinson's and Alzheimer's diseases, spinal cord injury, stroke, burns, heart disease, diabetes, osteoarthritis, and rheumatoid arthritis.

For example, it may become possible to generate healthy heart muscle cells in the laboratory and then transplant those cells into patients with chronic heart disease. Preliminary research in mice and other animals indicates that bone marrow stem cells, transplanted into a damaged heart, can generate heart muscle cells and successfully repopulate the heart tissue. Other recent studies in cell culture systems indicate that it may be possible to direct the differentiation of embryonic stem cells or adult bone marrow cells into heart muscle cells.

In people who suffer from type I diabetes, the cells of the pancreas that normally produce insulin are destroyed by the patient's own immune system. New studies indicate that it may be possible to direct the differentiation of human embryonic stem cells in cell culture to form insulin-producing cells that eventually could be used in transplantation therapy for diabetics.

To realize the promise of novel cell-based therapies for such pervasive and debilitating diseases, scientists must be able to easily and reproducibly manipulate stem cells so that they possess the necessary characteristics for successful differentiation, transplantation and engraftment. To be useful for transplant purposes, stem cells must be reproducibly made to: Proliferate extensively and generate sufficient quantities of tissue; Differentiate into the desired cell type(s); Survive in the recipient after transplant; Integrate into the surrounding tissue after transplant; Function appropriately for the duration of the recipient's life; Avoid harming the recipient in any way.

Recombinant trout E-peptides (i.e. rtEa2-, rtEa3-, and rtEa4-peptide) possess mitogenic activity in cultured BALB/3T3 fibroblasts and primary caprine mammary epithelium cell. In oncogenic transformed cell lines such as human breast cancer cells (MDA-MB-231), colon cancer cells (HT-29), neuroblastoma cells (SK-N-F1) and trout hepatoma cells, rtEa4- and hEb-peptides induced morphological differentiation and inhibited anchorage-independent cell growth. However, rtEa3-peptide showed no induction of morphological change and enhancement of cell attachment. In Salmon, Ea-1, Ea-3 and Ea-4 mRNA transcripts were detectable in the liver, and Ea-1 and Ea-3 levels increased dramatically in response to GH (growth hormone) treatment, whereas the amounts of Ea-4 mRNA was unchanged and most non-hapatic tissues expressed only the Ea-4 transcript, and expression was not influenced by GH, prolactin or somatolactin. Furthermore, rtEa4- and hEb-peptides have been shown to suppress the growth, invasion, and cancer cell induced angiogenesis of human breast cancer cells (MDA-MB-231) on chorioallantoic membrane (CAM) of developing chicken embryos (Chen and Chen, unpublished data).

Presently described are methods and compositions comprising IGF-I E-domain peptides with utility for modulating the differentiation of progenitor or stem cells. A method is described for using E-peptides, for example, the rtEa4-peptide of pro-IGF-I or human Eb-peptide of pro-IGF-I for modulating the differentiation of progenitor cells. The peptide species can be homolog of trout Ea4-peptide, human Eb-peptide (SEQ ID NO.:1) of pro-IGF-I or a fusion protein comprising the Ea4- or Eb-peptide of pro-IGF-I, and can be administered in a pharmaceutically acceptable composition alone or in combination with other compounds.

In an embodiment, the present invention provides a method for modulating the differentiation of a progenitor cell, comprising the step of treating a progenitor cell with an effective amount of a composition comprising an E-domain peptide or E-peptide.

In another embodiment, the present invention provides a method for inhibiting the differentiation of a hematopoietic progenitor cell, for example, a pluripotent or multipotent hematopoietic stem cell, comprising the step of treating a hematopoietic stem cell with an effective amount of a composition comprising an E-domain peptide or E-peptide. In certain embodiments, the peptide species comprises at least one of an rtEa4-peptide, a human Eb-peptide or a combination of both (e.g., either separately or linked, for example, chemically conjugated or linked contiguously in a single polypeptide chain).

In another embodiment, the present invention provides a method for promoting the differentiation of a neuronal stem cell, for example, a primary neuronal stem cell or a neuronal tumor progenitor cell, comprising the step of treating a neuronal stem cell with an effective amount of a composition comprising an E-domain peptide or E-peptide. In certain embodiments, the peptide species comprises at least one of an rtEa4-peptide, a human Eb-peptide or a combination thereof. In certain embodiments, the neuronal stem cell is a primary pituitary stem cell. In yet another embodiment, the neuronal stem cell is a neuroblastoma cell.

In another aspect, the invention relates to a progenitor cell that has been modified by treatment with an E-domain peptide of the invention. In certain embodiments, the invention relates to a cell comprising a hematopoietic stem cell that has been treated according to the methods of the invention.

In another embodiments, the invention relates to a cell comprising a neuronal progenitor cell that has been treated according to the methods of the invention. In certain embodiments, the neuronal stem cell is a primary pituitary cell. In certain other embodiments, the neuronal stem cell is a neuroblastoma cell.

As used herein, the term “stem cell” is used interchangeably with the term “progenitor cell”; both referring to cells that can give rise to at least one type of differentiated cell.

As used herein, the term “E-peptide” (or “E-peptide encoding nucleic acid”) or “E-domain peptide” (or “E-domain peptide encoding nucleic acid”) is used interchangeably to refer to the E-domain of an IGF-1 polypeptide or gene, respectively, of an animal, and portions thereof. In another aspect, the present invention contemplates a fusion protein comprising the amino acid or peptide sequence of an E-peptide or homologue of an E-domain of IGF-I, or a protein comprising the E-domain of IGF-I, fused or contiguous with a non-E-peptide. For example, in certain embodiments the invention includes fusion proteins comprising a “tag” or indicator portion and an E-peptide portion. In certain aspects the tag or indicator portion can be a peptide adapted for purification purposes, for example, FLAG tag, 6xHis tag, or the like. In other aspects, the tag peptide comprises a peptide adapted for providing a signal such as an antibody epitope or a fluorescent peptide. Still other aspects include the fusion of the E-peptide with another peptide that is adapted for mediating subcellular localization or translocation across a cellular membrane, for example, a TAT fusion protein from the HIV virus.

In another aspect, the invention relates to nucleic acid constructs (i.e., plasmids, vectors, expression constructs, and the like) comprising a nucleic acid encoding for an E-domain polypeptide or fragment thereof, operably linked with a DNA regulatory element, for example, a transcription, and/or replication regulatory element.

In certain aspects or embodiments described herein, the composition comprises at least one E-domain peptide having an amino acid sequence with at least about 30%, 40%, 50%, 60%, 70%, 80%, or 90% sequence homology with an hEb-domain peptide having the amino acid sequence of SEQ ID NO:1.

According to another aspect of the present invention, the peptide species is administered in a pharmaceutical composition comprising the E-peptide species and one or more pharmaceutically acceptable excipients, carriers, and/or adjuvants. In an alternative embodiment, the peptide species is administered to a progenitor cell by transforming the cells with exogenous nucleic acid that results in expression of an E-peptide in the cell.

In yet another embodiment, the present invention provides a method of modulating the differentiation of a cell comprising the step of administering to a progenitor cell a nucleic acid encoding a protein comprising an E-domain of IGF-I. Furthermore, the protein encoded by the nucleic acid administered according to the present invention comprises an a-type E domain or a b-type E domain of IGF-I, for example, rtEa4-peptide, hEb-peptide or a combination of both. Alternatively, the nucleic acid encodes a protein that is a homolog of the E domain of IGF-I, or a fusion protein comprising the E domain of IGF-I.

In another aspect, the invention provides methods for treating or preventing a disease, comprising the steps of isolating a stem cell from an individual, treating the stem cell in vitro or ex vivo, with an E-peptide, culturing the treated cell, and administering at least one of the treated cells to a patient.

Diseases or conditions that can be treated or prevented using therapeutic compositions and methods of the invention include, without limitation, e.g., cardiovascular disease, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, hypercoagulation, hemophilia, ulcers, wounds, lesions, cuts, abrasions, oxidative damage, age-related tissue degeneration, surgically related lesions, burns, muscle weakness, muscle atrophy, connective tissue disorders, idiopathic thrombocytopenic purpura, heart failure, secondary pathologies caused by heart failure and hypertension, hypotension, angina pectoris, myocardial infarction, tuberous sclerosis, scleroderma, transplantation, autoimmune disease, lupus erythematosus, viral/bacterial/parasitic infections, multiple sclerosis, autoimmune disease, allergies, immunodeficiencies, graft versus host disease, asthma, emphysema, ARDS, inflammation and modulation of the immune response, viral pathogenesis, aging-related disorders, Th1 inflammatory diseases such as rheumatoid arthritis, multiple sclerosis, inflammatory bowel diseases, AIDS, wound repair, heart attacks, heart failure, muscular dystrophy, bed sores, diabetic ulcers, oxidative damage, and tissue damage such as sinusitis or mucositis, wrinkles, eczema or dermatitis, dry skin, obesity, diabetes, endocrine disorders, anorexia, bulimia, renal artery stenosis, interstitial nephritis, glomerulonephritis, polycystic kidney disease, systemic, renal tubular acidosis, IgA nephropathy, nephrological diseases, hypercalceimia, Lesch-Nyhan syndrome, Von Hippel-Lindau (VHL) syndrome, trauma, regeneration (in vitro and in vivo), Hirschsprung's disease, Crohn's Disease, appendicitis, endometriosis, laryngitis, psoriasis, actinic keratosis, acne, hair growth/loss, allopecia, pigmentation disorders, myasthenia gravis, alpha-mannosidosis, beta-mannosidosis, other storage disorders, peroxisomal disorders such as zellweger syndrome, infantile refsum disease, rhizomelic chondrodysplasia (chondrodysplasia punctata, rhizomelic), and hyperpipecolic acidemia, osteoporosis, muscle disorders, urinary retention, Albright Hereditary Ostoeodystrophy, ulcers, Alzheimer's disease, stroke, Parkinson's disease, Huntington's disease, cerebral palsy, epilepsy, Lesch-Nyhan syndrome, multiple sclerosis, ataxia-telangiectasia, behavioral disorders, addiction, anxiety, pain, neuroprotection, Stroke, Aphakia, neurodegenerative disorders, neurologic disorders, developmental defects, conditions associated with the role of GRK2 in brain and in the regulation of chemokine receptors, encephalomyelitis, anxiety, schizophrenia, manic depression, delirium, dementia, severe mental retardation and dyskinesias, Gilles de la Tourette syndrome, leukodystrophies, cancers, breast cancer, CNS cancer, colon cancer, gastric cancer, lung cancer, melanoma, ovarian cancer, pancreatic cancer, kidney cancer, colon cancer, prostate cancer, neuroblastoma, and cervical cancer, neoplasm; adenocarcinoma, lymphoma; uterus cancer, benign prostatic hypertrophy, fertility, control of growth and development/differentiation related functions such as but not limited maturation, lactation and puberty, reproductive malfunction, and/or other pathologies and disorders of the like.

The compounds, nucleic acid molecules, polypeptides, and antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Alternatively, the present invention contemplates a method wherein the peptide species comprises a homolog of the E domain of IGF-I, or a fusion protein comprising the E domain of IGF-I. In addition, the present invention provides a method wherein the peptide species is administered in a pharmaceutical composition comprising the peptide species and one or more pharmaceutically acceptable excipients, carriers, or adjuvants. In an alternative embodiment, the injury repair occurs by transforming the cells with one or more exogenous nucleic acids that results in expression of an E-domain peptide of IGF-I in the cell.

In yet another embodiment, the present invention contemplates a method for promoting the differentiation of a stem cell, comprising administering to a cell at least one nucleic acid comprising a gene encoding a protein comprising an E-domain of IGF-I or portion thereof. Preferably, the encoded protein comprises a rtEa4-peptide. Alternatively, the protein comprises an E-domain of human IGF-I. Preferably, the protein comprises an Eb domain of human IGF-I. In another aspect, the protein comprises a homologue of the E-domain of IGF-I, or a fusion protein comprising the E-domain of IGF-I.

In other embodiments, the invention pertains to isolated nucleic acid molecules that encode E-peptides, E-peptide fusion proteins, and therapeutic compositions comprising the same.

The nucleic acids and peptides of the invention can be formed according to any of several well known methods, including, for example, using a nucleic acid or a peptide synthesizer according to standard methods. Alternatively, peptides of the invention can be formed by expressing a nucleic acid construct in a host cell (prokaryotic or eukaryotic) or cell extract, followed by an isolation and purification step.

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂, RbCl, liposome, or liposome-protein conjugate can be used. Transformation can also be performed after forming a protoplast of the host cell or by electroporation. These examples are not limiting on the present invention; numerous techniques exist for transfecting host cells that are well known by those of skill in the art and which are contemplated as being within the scope of the present invention.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. The eukaryotic cell may be a yeast cell (e.g., Saccharomyces cerevisiae) or may be a mammalian cell, including a human cell. For long-term, high-yield production of recombinant proteins, stable expression is preferred.

Oligonucleotides (eg; antisense, GeneBlocs) are synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204). The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163).

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above. The use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules and/or other chemical or biological molecules). The treatment of subjects with nucleic acid molecules can also include combinations of different types of nucleic acid molecules.

Recent attention has been focused on the biological activities of the proteolytically-processed polypeptides from post-translational modified peptide hormones. As discussed above, the C-peptide of pro-insulin has long been regarded to be biologically inactive except for a possible role in the folding of the insulin molecule during its post-translational modification. However, Ito et al. (1997) have reported that the C-peptide of pro-insulin was important in restoring vascular and neural dysfunction and Na+/K+-dependent ATPase activity in diabetic rats. Although a synthetic peptide amide of human b-type IGF-I E-peptide has been shown to exert mitogenic activity (Siefried et al., 1992), the biological activity of the native human E-peptides has not previously been identified.

Multiple alternative spliced forms of IGF-I transcript have been identified in mouse and rat (Roberts, et al., Mol. Endocrinol. 1: 243-48, 1987; Shimatsu, et al., J. Biol. Chem. 262: 7894-900, 1987). The alternative splicing of exon 5, resulting in variations in the E-domain of pro-IGF-I (Ea or Eb), has been shown to display developmental regulation and tissue specificity (Lin, et al, J. Endocrinol. 160: 461-67, 1999; Lin et al., Growth Horm IGF Res. 8: 225-33, 1998). Like mature IGF-I, as discussed in general above, the amino acid sequences of mouse or rat E-domains are highly homologous to their human counterparts. The biological significance of this conserved diversity of the E-domain and its differential expression is not clear. However, it is suggestive of potential biological activities associated with E-domain peptides. The presence of glycosylation sites on pro-IGF-I E-domains and the detection of such glycosylated products further suggest potential biological activity of E-domain peptides (Duguay, et al., J. Biol. Chem. 270: 17566-74, 1995). Indeed, a synthetic peptide amide with a 23-amino-acid sequence from the human pro-IGF-Ib E-domain (103-124) has been shown to possess mitogenic activity in human bronchial epithelial cells (Siegfried, et al., Proc. Natl. Acad. Sci. USA 89: 8107-11, 1992).

Thus, the present inventors have demonstrated that novel biological activities are associated with both the rainbow trout and human E-peptides.

The invention further includes a method for screening for a modulator of E-peptide activity. The method includes contacting a test compound with an E-peptide and determining if the test compound binds to said E-peptide. Binding of the test compound to the E-peptide indicates the test compound is a modulator of activity, or of latency or predisposition to the aforementioned disorders or syndromes.

The molecules of the instant invention can be used as pharmaceutical agents. Pharmaceutical agents prevent, inhibit the occurrence, or treat (alleviate a symptom to some extent, preferably all of the symptoms) a disease state in a subject.

In any of the embodiments described herein, the E-domain peptide can be combined with a pharmaceutically acceptable excipients, adjuvant, or carrier, a protein, lipid, glycol, glyceride, antioxidant, saccharide, or the like; another biologically active agent, for example, an analgesic or anti-inflammatory (e.g., aspirin, an NSAID, a COX inhibitor, or the like), an anesthetic, an anti-angiogenic (e.g., angiostatins or endostatin), a chemotherapeutic, a cytotoxic agent (e.g., antimetabolites, antibiotics, alkylating agense, alkaloids), an antineoplastic agent (e.g., cytokines, antibodies, vaccines), a hormonal agent (e.g., LHRH agonists, anti-androgens, anti-estrogens, aromatase inhibitors, progestagens), or the like.

In addition, the E-domain treatment in any of the embodiments described herein may be delivered via any pharmacological acceptable route, for example, oral, topical, anal, intravenous, enteral, parenteral, subcutaneous, intramuscular, transdermal, intracapsular, intraspinal, intracranial, or the like. Furthermore, in any of the embodiments described herein the E-domain peptide may be delivered in any pharmaceutically acceptable forms, for example, a powder, a liquid (e.g., a spray, intravenous solution), a gel, a polymeric matrix, a pill or capsule (e.g., a controlled release capsule, a time release capsule, or both), subdermal implant, and the like.

The term “host cell” includes a cell that might be used to carry a heterologous or exogenous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce one or more of the polypeptide components of the complex of the invention.

“Derivatives” are compositions formed from the native compounds either directly, by modification, or by partial substitution.

“Analogs” are nucleic acid sequences or amino acid sequences that have a structure similar to, but not identical to, the native compound.

Derivatives or analogs of the nucleic acids or proteins of the invention include, but are not limited to, molecules comprising regions that are substantially homologous to the nucleic acids or proteins of the invention, in various embodiments, by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS IN MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

“Homologs” can be naturally occurring, or created by artificial synthesis of one or more nucleic acids having related sequences, or by modification of one or more nucleic acid to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30% at the primary amino acid structure level, it is concluded that they share a common ancestor. For purposes of the present invention, genes are homologous if the nucleic acid sequences are sufficiently similar to allow recombination and/or hybridization under low stringency conditions.

As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA.

Furthermore, one of ordinary skill will recognize that “conservative mutations” also include the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, New York, 1989, and CURRENT PROTOCOLS IN MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47.

In yet another embodiment, a nucleic acid of the invention is expressed in mammalian cells using a mammalian expression vector. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

A polynucleotide can be a DNA molecule, a cDNA molecule, genomic DNA molecule, or an RNA molecule. A polynucleotide as DNA or RNA can include a sequence wherein T (thymidine) can also be U (uracil). If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to effect the desired process.

Transformation of a host cell with recombinant DNA may be carried out by conventional techniques as are well known to those skilled in the art. By “transformation” is meant a permanent or transient genetic change induced in a cell following incorporation of new DNA (i.e., DNA exogenous to the cell).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331 417, and Mesmaeker et al., 1994, Novel Backbone Replacements for Oligonucleotides, in Carbohydrate Modifications in Antisense Research, ACS, 24 39. These references are hereby incorporated by reference herein. Various modifications to nucleic acid (e.g., antisense and ribozyme) structure can be made to enhance the utility of these molecules. For example, such modifications can enhance shelf-life, half-life in vitro, bioavailability, stability, and ease of introduction of such oligonucleotides to the target site, including e.g., enhancing penetration of cellular membranes and conferring the ability to recognize and bind to targeted cells.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

Nucleic acid molecules of the invention can also be administered in the form of suppositories, e.g., for rectal administration of the drug or via a catheter directly to the bladder itself. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols. Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid. A pharmacological composition or formulation refers to a composition or formulation in a form suitable for administration, e.g., systemic administration, into a cell or subject, preferably a human. By “systemic administration” is meant in vivo systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By pharmaceutically acceptable formulation is meant, a composition or formulation that allows for the effective distribution of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutical composition of the invention is formulated to be compatible with its intended route of administration. Administration routes which lead to systemic absorption include, without limitations: parenteral, e.g., intravenous, intradermal, subcutaneous, oral (e.g., inhalation), transdermal (i.e., topical), transmucosal, intraperitoneal, inhalation, intrapulmonary, intrathecal, intramuscular, and rectal administration. In addition, there is provided a pharmaceutical formulation comprising a nucleic acid or peptide molecule of the invention and a pharmaceutically acceptable carrier. A molecule of the invention can be present in association with one or more non-toxic pharmaceutically acceptable carriers and/or diluents and/or adjuvants, and if desired other active ingredients. The pharmaceutical compositions of the invention can be in a form suitable for oral use, for example, as tablets, troches, lozenges, aqueous or oily suspensions, dispersible powders or granules, emulsion, hard or soft capsules, or syrups or elixirs.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Preparations for administration of the therapeutic of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the therapeutic complex of the invention) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Sustained-release preparations can be prepared. Suitable examples of sustained-release preparations include semipermeable matrices of solid hydrophobic polymers containing an E-peptide, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

A therapeutically effective dose refers to that amount of the therapeutic sufficient to result in amelioration or delay of symptoms. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the negatively charged polymer.

The amount of active ingredient that can be combined with the carrier materials to produce a single dosage form varies depending upon the host treated and the particular mode of administration. Dosage unit forms generally contain between from about 1 mg to about 1000 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The composition can also be administered to a subject in combination with other therapeutic compounds to increase the overall therapeutic effect. The use of multiple compounds to treat an indication can increase the beneficial effects while reducing the presence of side effects. In additional embodiments, the therapeutic of the invention may comprise one or more biologically active ingredients such as, Analgesics, Antacids, Antianxiety Drugs, Antiarrhythmics, Antibacterials, Antibiotics, Anticoagulants and Thrombolytics, Anticonvulsants, Antidepressants, Antidiarrheals, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Cold Cures, Corticosteroids, Cough Suppressants, Cytotoxics, Decongestants, Diuretics, Expectorants, Hormones, Hypoglycemics (Oral), Immunosuppressives, Laxatives, Muscle Relaxants, Sedatives, Sex Hormones, Sleeping Drugs, Tranquilizer, Vitamins or a combination thereof.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591 5; Kashani-Sabet et al., 1992, Antisense Res. Dev., 2, 3 15; Dropulic et al., 1992, J. Virol., 66, 1432 41; Weerasinghe et al., 1991, J. Virol., 65, 5531 4; Ojwang et al., 1992, Proc. Natl. Acad. Sci. USA, 89, 10802 6; Chen et al., 1992, Nucleic Acids Res., 20, 4581 9; Sarver et al., 1990 Science, 247, 1222 1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector.

In one aspect the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743 7; Gao and Huang 1993, Nucleic Acids Res., 21, 2867 72; Lieber et al., 1993, Methods Enzymol., 217, 47 66; Zhou et al., 1990, Mol. Cell. Biol., 10, 4529 37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3 15; Ojwang et al., 1992, Proc. Natl. Acad. Sci. USA, 89, 10802 6; Chen et al, 1992, Nucleic Acids Res., 20, 4581 9; Yu et al., 1993, Proc. Natl. Acad. Sci. USA, 90, 6340 4; L'Huillier et al., 1992, EMBO J., 11, 4411 8; Lisziewicz et al., 1993, Proc. Natl. Acad. Sci. U.S.A, 90, 8000 4; Thompson et al., 1995, Nucleic Acids Res., 23, 2259; Sullenger & Cech, 1993, Science, 262, 1566).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

The above referenced compositions are given by way of example and are not to be construed as limiting on the scope of the present claims. Indeed, the E-domain therapeutic of the present invention can be delivered in any number of pharmaceutically acceptable forms and routes, which will be readily apparent to those of ordinary skill in the art.

The present invention is further illustrated and described by the following examples, which are not intended to limit the scope of the invention in any way.

### EXAMPLE 1

### Cell Lines and Cell Culture Conditions

The following conditions were used for routine maintenance of cell cultures. Human breast cancer cells (MCF-7, ZR-75-1 and MDA-MB-231 cells) were obtained from American Type Cell Collection (ATCC, Rockville, Md.). They were cultured in F12/DMEM supplemented with 10% fetal bovine serum (FBS) and 10 ng/ml of insulin. Human colon cancer cells (HT-29 cells from ATCC) were cultured in F12/DMEM supplemented with 10% FBS; human HT1080 cells cultured in RPMI 1640 medium with 10% FBS; human hepatoma cells (HepG2 cells from ATCC), transformed human embryonic kidney cells (293GP cells, kindly provided by Dr. J. C. Burns at the University of California-San Diego) and human neuroblastoma cells (SK-N-F1 cells from ATCC) cultured in DME medium with high concentration of glucose and 10% FBS; and Poeceliposis lucida hepatoma cells (HC, kindly provided by Dr. Larry Hightower at the University of Connecticut) cultured in CO.sub.2-independent medium supplemented with 10% FBS. All cell cultures were incubated at 37.degree. C. under a humidified atmosphere of 5% CO.sub.2, except HC cells that were incubated at 30.degree. C. All tissue culture media and supplements used in this study were purchased from Gibco-BRL (Rockville, Md.).

Cells under various treatment conditions were maintained at 37.degree. C. in a 5% CO.sub.2 incubator and observed from 30 minutes to 72 hours after incubation (synthetic human Ea- and Eb-peptide were chemically synthesized at the Biotechnology Center, University of Connecticut). For treatment with synthetic hEa, hEb peptide or hlGF-I, 1.times.10.sup.5 cells were plated in each well of a 12-well culture plate in DMEM/F12 (1:1) supplemented with 0.4-3.2 .mu.M of synthetic hEa-, hEb-peptide and/or 5-10 nM of hlGF-I.

### EXAMPLE 2

### Transfection of Cells with a Construct Comprising Trout Ea-4 cDNA

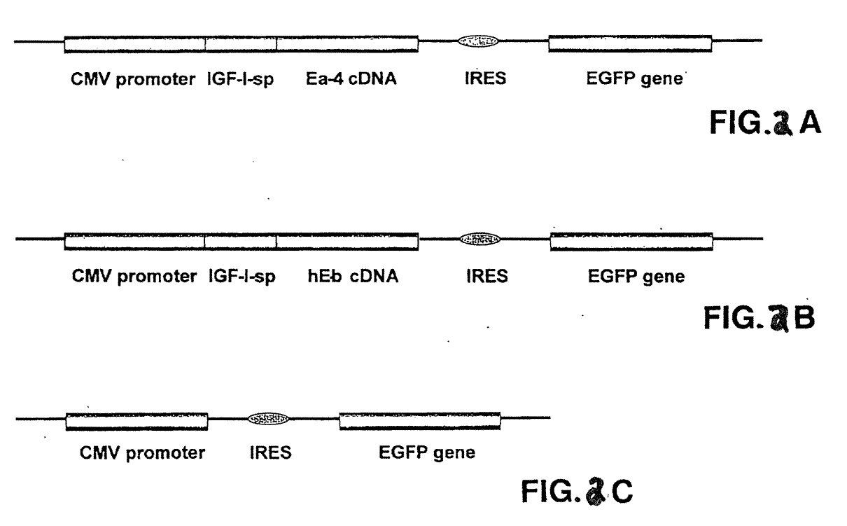

Two gene constructs, CMV-IGF-1-sp-Ea-4-cDNA-IRES-EGFP and CMV-IRES-EGFP were used in the transfection studies. The first construct (FIG. 2A) contained Ea-4 peptide cDNA (with a signal peptide sequence derived from hlGF-I), a ribosome re-entry site (IRES) and an enhanced green fluorescence protein (EGFP) marker gene. The other gene construct (FIG. 9C) contained IRES and EGFP, but did not contain Ea-4-peptide cDNA. The expression of both gene constructs was driven by a promoter from CMV. Vectors containing the recognized control sequences and marker gene are available from commercial sources (CLONTECH, Palo Alto, Calif.).

Transfection of the cells was accomplished as follows. MDA-MB-231 cells were cultured in F12/DMEM supplemented with 10% FBS and 10 ng/ml of insulin to 90% confluence. About 5.times.10.sup.6 cells were harvested and resuspended in 1 mL of serum-free F12/DMEM containing 20 .mu.g of un-linearized constructs. The cells were electroporated in a BRL Cell-Porator using the following settings: low .OMEGA., 1180 micro Faraday (.mu.F) capacitance, and two pulses at 200 volts. Following electroporation, cells were resuspended in 12 mL of fresh growth medium and seeded into a 6-well plate to recover. Permanent transfectants expressing green fluorescence protein (GFP) were enriched in a medium containing neomycin (G418) at 1 mg/mL for ten days and followed with 500 μg/mL for continuous maintenance. Individual green cell clones of transfectants were isolated from the enriched population by the method of serial dilution.

The presence of the transgene and the expression of Ea-4 (SEQ ID NO: 2) in transfectants were determined by PCR and comparative RT-PCR assays following conditions described by Greene et al. (1999). Ea-4-peptide specific primers used in the amplification were: forward primer (5′-CTTGTGGCCGTTTACGTC-3′) (SEQ ID NO 6); AND reverse primer (5′-GCACAGCACCCAGACAAG-3′) (SEQ ID NO 7).

Results of PCR analysis of genomic DNA isolated from transfectants confirmed that clones E-9 and E-15 contained Ea-4-peptide cDNA, whereas control EGFP clones did not. Comparative RT-PCR analysis showed that about same levels of mRNA for Ea-4-peptide were detected in both clones E-9 and E-15, while no Ea-4 mRNA was detected in EGFP control clones. Soft Agar Colony Formation assay showed that while EGFP-transfected MDA-MB-231 cell clones formed colonies on soft agar medium, none of the Ea-4-peptide gene transfected MDA-MB-231 clones (ie., E9 and E15) form any colonies in soft agar medium, suggesting that the colony formation activity of MDA-MB-231 cells is abolished by the Ea-4 peptide. Furthermore, obvious morphological changes were also observed in MDA-MB-231 transfected cells expressing the Ea-4-peptide gene (FIG. 10E).

Because the signal peptide sequence of human IGF-I was also included in the Ea-4 cDNA transgene, the Ea-4 peptide produced by the transfected cell clones would be secreted into the medium. To confirm this, media isolated from EGFP clone and E15 clone were tested for their activities to induce morphological change in untransfected MDA-MB-231 cells. Results presented in FIGS. 10F, 10G and 10H showed that while medium isolated from E15 clone was able to induce the morphological change of untransfected MDA-MB-231 cells, medium isolated from EGFP cells could not. Therefore, these results rule out the possibility that any contaminant from the E. coli extract could result in the anti-tumor activity observed above.

### EXAMPLE 3

### Transfection of Cells with a Construct Comprising hEb cDNA

Two gene constructs, CMV-IG F-1-sp-hEb-cDNA-IRES-EGFP and CMV-IRES-EGFP were used in the transfection studies. The first construct (FIG. 2B) contained hEb peptide cDNA (with a signal peptide sequence of hlGF-1), a ribosome re-entry site (IRES) and an enhanced green fluorescence protein (EGFP) marker gene. The other gene construct (FIG. 2C) contained IRES and EGFP, but did not contain hEb-peptide cDNA. The expression of both gene constructs was driven by a promoter from CMV.

Transfection of the cells was accomplished as follows. MDA-MB-231 cells were cultured in F12/DMEM supplemented with 10% FBS and 10 ng/ml of insulin to 90% confluence. About 5.times.10.sup.6 cells were harvested and resuspended in 1 mL of serum-free F12/DMEM containing 20 μg of un-linearized constructs. The cells were electroporated in a BRL Cell-Porator using the following settings: low •, 1180 micro Faraday (μF) capacitance, and two pulses at 200 volts. Following electroporation, cells were resuspended in 12 mL of fresh growth medium and seeded into a 6-well plate to recover. Permanent transfectants expressing green fluorescence protein (GFP) were enriched in a medium containing neomycin (G418) at 1 mg/mL for ten days and followed with 500 μg/mL for continuous maintenance. Individual green cell clones of transfectants were isolated from the enriched population by the method of serial dilution.

The presence of the transgene and the expression of hEb in transfectants were determined by PCR and comparative RT-PCR assays following conditions described by Greene et al. (1999). hEb-peptide specific primers used in the amplification were: forward primer (5′-CTTGTGGCCGTTTACGTC-3′) (SEQ ID NO 6); AND reverse primer (5′-GCACAGCACCCAGACAAG-3′) (SEQ ID NO: 7).

### EXAMPLE 4

### Morphological Changes Induced by Rainbow Trout Ea-4 Peptides and Synthetic Human Analogues

Approximately 1-2×10⁵ of MCF-7, ZR-75-1, HT-29, HepG2, 293GP, HC or SK-N-F1 cells re-suspended in their respective basal medium without fetal bovine serum (FBS) were plated in a 6-well culture chamber. Prior to plating cells, an acid-washed coverslip was placed in each well of the culture chamber. Recombinant rainbow trout E-peptides (rtEa-2, rtEa-3 or rtEa-4 peptide at 0.8 μM), human IGF-I (hIGF-1, 2.5 nM) or the same amount of control protein was added to each well and the cell cultures were incubated at 37° C. under a humidified atmosphere of 5% CO₂. The control protein was prepared from E. coli cells carrying the expression plasmid but without the E-peptide gene according to the purification method described by Tian et al. (1999). Coverslips were removed from the culture chamber 24 hours after initiation of the treatment and observed under an Olympic inverted microscope equipped with differential interference phase contrast objective lenses or phase contrast objective lens (final magnification, 200×). The morphological change assay was performed at least 10 times with different batches of Ea-4 peptide preparations. The concentration of these synthetic peptides tested was 0.4 μM.

### EXAMPLE 5

### Effect of Inhibition of mRNA and Protein Synthesis on Morphological Changes

To study the effects of alpha-aminitin and cycloheximide, known inhibitors of RNA and protein synthesis, respectively, on morphological changes induced by rtEa-4-peptide, about 1-2×10⁵ of ZR-75-1 and 293GP cells, re-suspended in their respective basal medium without FBS, were plated in a 6-well culture chamber. Prior to plating the cells, an acid-washed coverslip was placed in each well of the culture chamber. Each culture was treated with recombinant trout Ea-4-peptide at 0.8 μM, and with either alpha-aminitin at 10 μg/mL (an RNA synthesis inhibitor) or with cycloheximide at 1.0 μg/mL (a protein synthesis inhibitor). The cell cultures were incubated at 37° C. under a humidified atmosphere of 5% CO₂. Coverslips were removed from the culture chamber 24 hours after initiation of the treatment and observed under an Olympic inverted microscope equipped with differential interference phase contrast objective lenses (final magnification, 200.times.). The viability of the inhibitor-treated cells was further determined by a dye extrusion assay.

To determine whether the morphological changes induced by the Ea-4 peptides requires synthesis of new proteins or of RNA, 293GP and MCF-7 cells were cultured under the same conditions as described above. Ea-4 peptide-induced morphological changes in 293GP and MCF-7 cells were abolished by treatment with cycloheximide or alpha-aminitin. These results suggest that the morphological change induced by the Ea-4 peptide might result from expression of genes that were activated and/or inactivated during oncogenic transformation or tumor development. This conclusion is further substantiated by results of studies on microarray screening of a collection of human EST's that indicate that the Ea-4 peptide up- and/or down-regulated the expression of a series genes related to cell attachment, proliferation and invasion of MDA-MB-231 cells.

### EXAMPLE 6

### Relative Activity of Trout Ea-2, Ea-3, and Ea-4 Peptides

In examining the biological activity of E-peptides of human pro-IGF-1, the present inventors have determined that hEb peptide (SEQ ID NO: 1), like rainbow trout Ea-4 SEQ ID NO:2) peptide, evidences novel and unique activities, apart from the know functions of mature IGF-1. The in vitro effective concentration of synthetic hEb peptide (0.4-3.2 •M) is within a similar range as that of the recombinant rtEa-4 peptide.

Cells were again cultured as described above. Twenty-four hours after treatment with 0.8 μM of E-peptides or the control protein, the cells were observed under an Olympic inverted microscope equipped with differential interference phase contrast objective lenses (200× magnification). Although both Ea-2- and Ea-4-peptides were able to induce morphological change in 293GP or ZR-75-1 cells, the Ea-3 peptide failed to induce any visible morphological change under the identical culture conditions. This observation indicates that the domain of the E-peptide responsible for the induction of morphological change in the 293GP or ZR-75-1 is not present in the Ea-3-peptide. To confirm this hypothesis, synthetic peptides specific to Ea-1-, Ea-2-, Ea-3- and Ea-4-peptide (Ea-1sp, Ea-2sp, Ea-3sp and Ea-4sp) specific sequence (see FIG. 1) were prepared and tested for their activities to induce morphological change in ZR-75-1 cells. Cells were cultured in F12/DME medium without FBS as described above. Twenty-four hours after treatment with 0.8 μM of synthetic peptide containing Ea-1 sp, Ea-2sp, Ea-3sp or Ea-4sp specific sequence or the control protein, the cells were observed under an Olympic inverted microscope equipped with differential interference phase contrast objective lenses (200× magnification). Ea-2sp and Ea-4sp, but not Ea-1sp and Ea-3sp at 0.4 μM were able to induce a morphological change. These results indicate that the active domain of the Ea-peptide resides within the 12 amino acid residues of Ea-2-peptide.

### EXAMPLE 7

### Morphological Changes and Inhibition Assays Using Human Eb Peptide

Neuroblastoma SK-N-F1 cells (10⁵) were seeded into 12-well culture plates in DMEM/F12 (1:1) supplemented with 0-3.2 μM of synthetic hEb-peptide, or buffer control, and incubated at 37° C. in a 5% CO₂ humidified incubator.

For inhibition studies, cells were pre-incubated with vehicle (0.1% DMSO), or 10-50 μM of the MEK inhibitor PD98059 (Promega, Madison, Wis.), or 10 nM-1 μM of the PI-3K inhibitor wortmannin (Sigma), or 10-50 μM of LY294002 (Promega, Madison, Wis.), for one hour prior to the addition of 3.2 μM hEb-peptide.

Cell images were taken by random sampling at various time points using a MicroMAX CCD camera (Princeton Instruments, Bozeman, Mont.). Approximately 1000 cells were analyzed from each treatment, carried out in triplicate. Cells with neurites longer than one cell body diameter (>20 μm for SK-N-F1 cells) were scored as positive neurite-bearing (Fagerstrom, et al., Cell Growth Differ., 7: 775-85, 1996; Morrione, et al., Cancer Res. 60: 2263-72, 2000). The percentage of neurite-bearing cells and the respective length of neurites were measured with reference to a stage micrometer and analyzed using the public domain NIH Image program.

### EXAMPLE 8

### Morphological Differentiation (Neurite-Like Growth) in Neuroblastoma Cells

SK-N-F1 neuroblastoma cells are characterized as poorly differentiated embryonal cells with an epithelial-like morphology. In examining the biological activities of synthetic hEa and hEb peptides of pro-IGF-I, the present inventors determined that hEb peptides induce morphological changes in SK-N-F1 cells, whereas synthetic hEa-peptide, like mature IGF-I, lacks this activity. Cells treated with the mature hlGF-I (5 nM) remained rounded and formed aggregated clusters, similar to the morphology of the control cells, and that of the cells treated with 3.2 μM synthetic hEa-peptide alone, or in combination with 5 nM hlGF-I. In contrast, SK-N-F1 cells treated with synthetic hEb-peptide (1.6 μM or 3.2 μM) differentiated into a neuron-like morphology with one or multiple neurite-like processes and a relatively small cell body. The treatment of mature hlGF-I and hEb-peptide combined further enhanced the formation of neurite-like processes, and resulted in a 20-30% increase in the percentage of neurite-bearing cells at 1-4 hours after stimulation (quantitative data not shown). The activities of hEb-peptide are identical to those of rainbow trout Ea-4-peptide described above.

To further characterize the dose-response relationship and time course of hEb peptide in inducing neurite-like process outgrowth, SK-N-F1 neuroblastoma cells were treated with various amounts (0 to 3.2 μM) of hEb peptide over a course of 72 hours. Images of cells were taken by random sampling after 1 h, 6 h, 24 h, 48 h and 72 h of incubation. The average length of neurite-like process outgrowth was measured by random sampling of more than 1000 cells at various time points. The neurite-like processes started to be visible as early as 0.5-1 hour after the addition of hEb peptide. The effect of hEb peptide in inducing neurite-like outgrowth was dose-dependent, as evident from a comparison of cells treated with 0.4 μM hEb peptide with those treated with 0.8-3.2 μM hEb peptide over 24-72 hours. The maximum effect of hEb peptide was observed 48 hours after the addition of 3.2 μM hEb peptide, with an average neurite length of 50 to 60 μm (Table 2, below).

TABLE 2
Dose Response and Time Course Studies on hEb-Peptide Induced
Neurite Growth.

| [hEb- | | | | | |
|---|---|---|---|---|---|
| peptide] | Neurite length (μm)^{‡} | | | | |
| (μM) | 2 h | 6 h | 24 h | 48 h | 72 h |
| 0 | 19 ± 0.1 | 21 ± 4.6 | 24 ± 2.2 | 24 ± 0.3 | 24 ± 1.3 |
| 0.4 | 30 ± 1.6 | 31 ± 7.6 | 29 ± 5.2 | 35 ± 0.9 | 32 ± 3.1 |
| 0.8 | 33 ± 2.1 | 36 ± 0.6 | 38 ± 3.9 | 43 ± 1.0 | 41 ± 2.5 |
| 1.6 | 33 ± 3.8 | 38 ± 3.5 | 39 ± 1.8 | 47 ± 3.9 | 48 ± 2.8 |
| 3.2 | 33 ± 2.8 | 39 ± 2.2 | 45 ± 1.7 | 52 ± 3.7 | 48 ± 2.7 |

^{‡}neurite length shown as mean ± standard deviation determined from more than 1000 cells sampled in triplicate at each time point; a dose-response relationship was observed when comparing cells treated with 0.4 μ.M hEb-peptide and those treated with 0.8-3.2 μM hEb-peptide with statistical differences (P · 0.05) from 24-72 hours; the maximum effect of hEb-peptide was obseved at 48 h after initiation of treatment.

In examining the biological activity of E-peptides of human pro-IGF-1, the present inventors have determined that hEb peptide, like rainbow trout Ea-4 peptide, evidences novel and unique activities, apart from the known functions of mature IGF-I. The in vitro effective concentration of synthetic hEb peptide (0.4-3.2 .mu.M) is within a similar range as that of the recombinant rtEa-4-peptide.

### EXAMPLE 9

### Effects of Trout Ea-4 Peptide on Colony Formation

An obvious change in the characteristics of normal cells after oncogenic transformation is the loss of contact inhibition and anchorage-dependent cell division behavior (Kosaki et al., 1999). This behavioral change in oncogenic transformed or established cancer cells can be easily demonstrated in vitro by a colony formation assay in a semi-solid medium (Dickson et al., 1986).

Colony formation assays were conducted following the method described by Yang (1975). About 2×10⁴ of HT-29 (colon cancer cells) or MDA-MB-231 cells (aggressive breast cancer cells) at log phase were plated in their respective basal medium containing 1.25% FBS and 0.5% purified agar (Difco laboratories), and supplemented with various concentrations (0.4 to 1.6 μM) of the recombinant rainbow trout Ea-4 peptide, or the same amount of the control protein, in 6-well culture chambers. After the medium is solidified, each well is overlaid with 1 ml of the basal medium (containing 1.25% FBS) supplemented with same concentration of the trout Ea-4 peptide. The plates were incubated at 37° C. in a humidified incubator with 5% CO₂ and examined daily under an inverted microscope for 2-3 weeks. Colonies were observed under an Olympic inverted microscope equipped with phase contrast objective lenses (final magnification: 40×). Colonies with sizes •50 μm were scored. The viability of cells at the conclusion of the experiment was confirmed by dye extrusion assay with tryptan blue. The assay was conducted two times.

To confirm whether treatment of transformed cells with the Ea-4 peptide could result in increased attachment of the cells to the culture dish, 293GP cells were cultured in a serum-free basal medium supplemented with Ea-4-peptide (0.8 μM) or 10% FBS, respectively, in 6-well culture chambers. After four days, the culture medium was removed, and cells were rinsed twice with PBS containing 0.02% EDTA, fresh PBS was added, and the culture plates shaken 20 times manually. At the end of shaking, cells cultured in serum-free medium or medium supplemented with FBS detached completely from the culture chamber, while cells cultured in the serum-free medium supplemented with Ea-4 peptide remained attached to the culture chamber. These results clearly showed that rtEa-4 peptide enhances the attachment of oncogenic transformed cells to the culture chamber, similar to the behavior exhibited by untransformed (normal) cells.

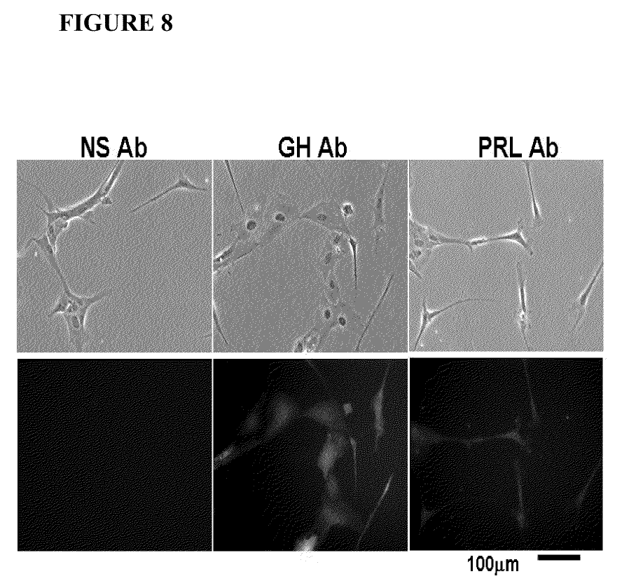

It has been suggested that the malignant growth property of human neuroblastoma cells can be associated with their differentiation status (Martin, et al., J. Pediatr. Surg. 3: 161-64, 1968). Spontaneous resolution has in fact been observed as a result of neuronal differentiation of neuroblastoma cells in vivo (Pahiman, et al., Eur. J. Cancer., 31A: 453-58, 1995). As shown in FIGS. 8A and 8B, many visible colonies were developed from both cancer cell lines grown in the soft agar medium supplemented with 1.25% FBS and the control protein, but fewer colonies were developed from both cell lines cultured in the same medium supplemented with increasing concentrations of recombinant Ea-4 peptide. These results showed that Ea-4 peptide is able to reduce or abolish the anchorage-independent cell division behavior of tumor cells.

### EXAMPLE 10

### Effects of hEb Peptide on Colony Formation

Poor differentiation and anchorage-independent cell growth are among the hallmarks of poor prognosis in neuroblastoma disease. As discussed above, neuroblastoma cells present a unique system in which the relationship between differentiation and tumorigenesis might be successfully dissected. Loss of proper differentiation is a common theme in cellular transformation in many different types of cancer. Thus, inducing cellular differentiation and intervening growth factor signaling have now been discussed as novel alternative approaches to cancer treatment (Garattini and Terao, Curr. Opin. Pharma., 1: 358-63, 2001; Favoni, de Cupis, Pharmcol. Rev., 52: 179-206, 2000). According to the present invention, hEb peptide, like rainbow trout Ea-4 peptide, induces morphological differentiation in neuroblastoma cells.

The effect of hEb-peptide on in vitro colony formation was tested in the presence or absence of either the mature hlGF-I or fetal bovine serum. Human neuroblastoma SK-N-F1 cells (10⁴) were mixed with 0.4% soft agar supplemented with or without hEb peptide (3.2 μM), IGF-I (5 nM) and/or fetal bovine serum (2.5%), as indicated. The cell mixtures were seeded on top of a solidified basal medium DMEM/F12 (1:1) containing 0.5% agar. Medium supplemented with various peptides or serum were overlaid on top of the solidified cell layer followed by a two-week period incubation at 37° C. in a 5% CO₂ humidified incubator. The percentage of cells formed into colonies with a diameter greater than 100 μm Macpherson, Tissue culture methods and applications, pp276-80: N.Y. Academic Press, 1973) were scored in triplicate and subjected to Student t-test analysis. At least three independent assays under each treatment conditions were carried out.

Mature hlGF-I (5 nM), like fetal bovine serum (FBS, 2.5%) strongly stimulated colony formation in neuroblastoma cells (SK-N-F1). On the other hand, hEb-peptide (3.2 μM) significantly reduced the percentage of cells grown into colonies with a diameter greater than 100 μm. In the absence of serum and growth factors, inhibition of colony formation by hEb peptide was 64%, similar to that in the presence of hlGF-I (5 nM) (59% inhibition). Colony formation in the presence of FBS (2.5%) was inhibited by 73%. According to the present invention, in a manner similar to that demonstrated for rtEa-4 peptide, the hEb peptide of human pro-IGF-I exhibits an inhibitory effect on anchorage-independent growth by 59-73%. This activity is in sharp contrast to the stimulatory effect of mature IGF-I.

To further confirm the effect of hEb-peptide on reduction or elimination of malignant growth of cancer cells, aggressive breast cancer cells, MDA-MB-231 and ZR-75-1 cells, were transfected with a hEb-peptide gene construct. hEb gene transfected cells, namely hEb A, hEb B, hEb C and hEb H, exhibited a morphology similar to that of untransfected MDA-MB-231 treated with synthetic hEb-peptide. Furthermore, results of the colony formation assay showed that, like treatment of cancer cells with synthetic hEb-peptide, the colony formation activities of hEb gene transfected cells were greatly reduced or diminished completely.

Normally, adherent cells require anchorage to extracellular matrix (ECM) to survive and proliferate. This anchorage dependency is primarily mediated by integrins that are responsible for engaging cell-ECM interaction and thus activating the growth- and survival-promoting signals. Tumor cells, including neuroblastoma cells, are generally resistant to apoptosis induced by loss of attachment to ECM and cannot only survive but grow independently of anchorage. According to the present invention, the hEb peptide of human pro-IGF-I restores the anchorage dependency for cell survival and cell division in neuroblastoma cells. These results suggest, without limiting the present invention, that hEb-peptide induced signaling may act collaboratively and converge with extracellular adhesion signaling pathways in regulating cell survival and division. The results provided herein also indicate that the hEb peptide, but not the hEa peptide of human pro-IGF-I induces morphological differentiation and inhibits anchorage-independent growth in human neuroblastoma cells. A similar nature and range of biological activities have been shown with Ea-4 peptide of rainbow trout pro-IGF-I. Thus, E-peptides of pro-IGF-I are not only biologically active but are functionally conserved in fish and humans. Furthermore, the data disclosed herein also indicate, without limiting the scope of the present invention, that these conserved E-peptide activities might be mediated by conserved signal transduction mechanisms.

### EXAMPLE 11

### Invasion Assays

An obvious characteristic of cancer cells is their ability to invade normal tissues (metastasis) by migrating to other locations and subsequent colonization. The molecular events of metastasis have become clearer in recent years. These events involve the secretion of metalloproteases by tumor cells, digestion of basement membrane (invasion), and migration and colonization of cancer cells in new locations (Clezardin 1998). The invasive behavior can be demonstrated by an in vitro invasion assay where the migration of cancer cells across a semi-solid Matrigel (proteins isolated from basement membranes) is measured. To investigate whether the Ea-4-peptide of trout pro-IGF-1 can retard the invasive activity of cancer cells, an in vitro invasion assay was conducted in HT1080 cells, a known invasive cancer cell line, in the presence of Ea-4-peptide.

Invasion assays were conducted in BIOCOAT MATRIGEL invasion chambers following the procedure provided by Becton Dickinson Labware (Bedford, Mass.; 40480 and 40481 guidelines). According to these procedures, 1×10⁶ of HT1080 cells in DMEM supplemented with 1.25% FBS, with Ea-4 peptide (0.17 μM and 0.34 μM), or the same amount of the control protein, were plated in each insert of the Matrigel or control invasion chambers. The inserts were placed in the respective chambers containing DMEM medium supplemented with 10% FBS, and the chambers were incubated at 37° C. under a humidified atmosphere of 5% CO₂ for 24 hours. After removal of the non-invaded cells with cotton swabs, the invaded cells on the other side of the membranes were stained with the Diff-Quick T stain (Becton Dickinson Labware, Bedford, Mass.) and observed under an Olympic inverted microscope (magnification, 200.times.). Control proteins were prepared from E. coli cells carrying the expression plasmid without the Ea-4-peptide gene by the same purification method (Tian et al., 1999). The assay was repeated three times.

As shown in Table 3, below, treatment of HT-1080 cells with trout Ea-4-peptide results in a dose-dependent reduction of the invasive activity of HT 1080 cells.

TABLE 3
Effect of Ea4-Peptide on The Invasion Activity of HT-1080 Cells¹

| | #invaded cells/view | #invaded cells/view | | % Reduction |
|---|---|---|---|---|
| Treatment | (MIC)² | (CIC)³ | % Invasion⁴ | of invasion |
| No Ea-4 | 63 ± 13 | 157 ± 6 | 40 | 0 |
| Control | 62 ± 5 | 157 ± 6 | 39 | 2 |
| Ea-4 (0.17 μM) | 30 ± 5 | 157 ± 6 | 19 | 52 |
| Ea-4 (0.34 μM) | 24 ± 1 | 157 ± 6 | 15 | 62 |

¹Assay conducted in BIOCOAT MATRIGEL invasion chambers following procedure provided by Becton Dickson Labware (Bedford, MA, guidelines #40480 and #40481).
²MIC: mean number of invaded cells per view invaded throughMatrigel insert membrane; each cell number determined as average of three independent counting; reported ± standard deviation of the mean.
³CIC: mean number of cells pre view migrated through control insert membrane; each cell number determined as average of three independent counting; reported ± standard deviation of the mean.
⁴% invasion = mean # cells invading through Matrigel insert membrane/mean # cells invading through insert membrane.

### EXAMPLE 12

### Anti-Angiogenesis Activity of the Trout Ea-4 Peptide

The term, angiogenesis, as used herein, refers to the generation of new blood vessels in a tissue or an organ. Under normal physiological conditions, angiogenesis is invoked under controlled, specific situations. In disease states, however, the control is altered and pathological damage can occur. It is known that the growth and spread of solid tumors, such as breast cancer, depends on angiogenesis. In view of the role of angiogenesis in cancer and other diseases, it is desirable to have a means of reducing or inhibiting the process. It is hoped that anti-angiogenetic agents will stop the growth of cancer cells by blocking the blood supply and thus preventing the formation of new vessels that feed the cancerous cells. The activity of the peptides on angiogenesis were compared to a known anti-angiogenetic agent, endostatin. Endostatin, a proteolytic cleavage product of type XVIII collagen, is a potent angiogenesis inhibitor. The protein is a specific inhibitor of endothelial proliferation and angiogenesis, as described in U.S. Pat. No. 5,854,205, hereby incorporated by reference.

A suitable assay is the chick embryo chorioallantoic membrane (CAM) assay described by Crum et al. Science 230:1375 (1985). See also, U.S. Pat. No. 5,001,116, hereby incorporated by reference, which describes the CAM assay. Briefly, the Ea-4 peptide (40 μg and 80 μg), endostatin (5 μg and 10 μg) and a PBS control buffer were delivered onto the chorioallantoic membrane (CAM) of three-day old chick embryos. The CAMs were photographed in ovo with a digital camera on day 7. The angiogenic response was assessed by counting the number of intersect points of the blood vessels spread out in a defined field (vessel density). The Ea-4 peptides in habited blood vessel branching.

### EXAMPLE 13

### Anti-Angiogenesis Activity of the hEb Peptide

Each were dissolved in PBS, and various and known amounts of hEb-peptide (250 μg, 500 μg and 1000 μg respectively) and human endostatin (10 μg and 20 μg respectively) were applied to the CAM. Pictures were taken on day 7 for vessel density determination. The extent of inhibition of angiogenesis on CAM was scored from the defined area of CAM. The anti-angiogenic effect of hEb-peptide was measured on chorioallantoic membranes of chick embryos. The hEb-peptide exerts a dose-dependent reduction of vessel density in the chorioallantoic membrane of chicken embryos.

### EXAMPLE 14

### Inhibition of Hematopoiesis

This invention relates the use of trout Ea4-peptide or human Eb-peptide of pro-IGF-I to inhibit the differentiation of hematopoietuc stem cells and stimulate the differentiation of neuronal stem cells. In particular, the present invention relates the use of IGF-I E peptides for suppressing malignant growth of neuroblastoma cells and inducing both neuroblastoma cells and neuronal stem cells to go into differentiation. Specific evidence leading to this invention is listed below:

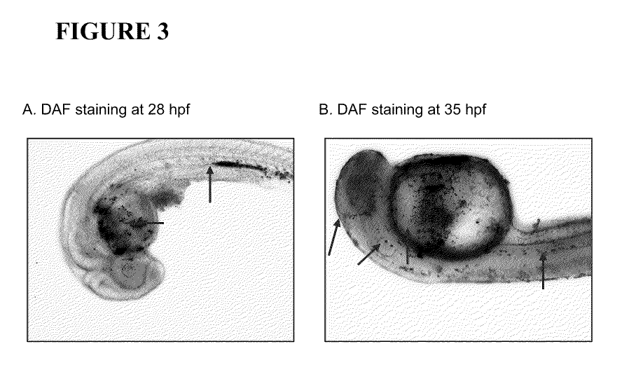

FIG. 3 shows the effect of rtEa4-peptide on inhibition of early embryonic red blood cell development in zebrafish embryos. Details of the experiment: Zebrafish embryos at 2.5 hours after fertilization (hpf) were injected with 1.0 pmole of rtEa4-peptide or control peptide, fixed at 36 hpf and stained with diaminofluorene for visualization of red blood cells. The results showed that rtEa4-peptide inhibited the development of embryonic red blood cells. Same results were observed when the embryos were injected with hEb-peptide. These results suggest that rtEa4-peptide or hEb-peptide may inhibit hematopoiesis in zebrafish embryos.

Table 6 shows the results of the suppression of specific hematopoietic gene expression by rtEa4-peptide or hEb-peptide. Details of the experiment: Data presented in FIG. 3 showed that E-peptide inhibits the differentiation of hematopoietic stem cells into primitive hematopoietic progenitor cells that lead to the production of early embryonic red blood cells. It is equally possible that E-peptide may also inhibit the differentiation of heamatopoietic stem cells into definitive hematopoietic stem cells leading to production of erythrocytes, lymphocytes, monocytes/macrophages and neutrophils. To prove this hypothesis, the effect of E-peptide on expression of essential hematopoietic genes was determined by quantitative real-time RT-PCR analysis. Total RNA samples were prepared from zebrafish embryos treated with E-peptide and control peptide by the guanidinium thiocyanate-phenol-chloroform method. Levels of gata 1, gata 2, fli 1a, c-myb, i-plastin and ikaros mRNAs were determined by quantitative real-time RT-PCR method and data expressed as: Fold of reduction=1/2−(••CT), where ••CT is •CT sample −•CT control. Each data point was the average of at least 6 determinations. Results of the analysis showed that rtEa4- or hEb-peptide of pro-IGF-I suppresses the expression of gata 1, gata 2, fli 1a, c-myb, i-plastin and ikaros genes. These results prove the hypothesis that E-peptide inhibits hematopoiesis.

Materials & Methods

Fish and embryos. Zebrafish were raised according to standard methods in a recirculating system (the Stand-Alone System, Aquatic Habitats, Benbow Court Apopka, Fla.). At the onset of light, female and male fish were put together in a ratio of 1:1 in an embryo collector tank at 28.5° C., and fertilized embryos were collected within 30 min of mating and used for microinjection.

Microinjection of recombinant rtEa4-peptide. Recombinant rtEa4-peptide was prepared by following the method described, previously. As control proteins, rtEa3-peptide was synthesized at the Biotechnology Center in the University of Connecticut, Storrs, Conn. and bovine serum albumin (BSA) was purchased from Invitrogen (Carlsbad, Calif.). One pmol of rtEa4-, rtEa3-peptide and BSA in 0.25 M KCl with 0.01% (v/v) phenol red were ready to be injected prior to microinjection. All embryos were closely monitored following embryonic development under a dissecting microscope (Olympus SZX12, Japan). Ten nanoliter of injection solution was microinjected into an embryo using a Nanoliter Injector (World Precision Instruments Co., Sarasota, Fla.) at 2.5 hpf. The microinjected eggs were cultured at 28.5° C. in an embryo medium. Embryos were dechorinated with fine forceps and subjected to Diaminofluorene staining.

Hemoglobin analysis by Diaminofluorene (DAF) staining. Normal or defective zebrafish embryos were dechorinated prior to staining. A DAF stock solution was made by dissolving DAF (500 mg) in 10 ml of glacial acetic acid (90%) with vigorous vortexing. For hemoglobin detection, a working solution was prepared by mixing 50 μl of DAF stock solution with 50 μl of 30%, hydrogen peroxide and 5 ml of 200 mM, Tris-HCl, pH 8.0. The dechorinated embryos were submerged into 7 ml of DAF working solution in a Petri-dish (60×150 mm). After incubation for 20 min in dark, embryos were washed 3 times with phosphate-buffered saline (PBS) and examined for dark-blue stained hemoglobin under a dissecting microscope (Olympus SZX12, Japan). Taken images were stacked using the NIH ImageJ software (http://rsb.info.nih.gov/ij) to generate multi-focus image.

Conventional RT-PCR and relative quantitative real-time RT-PCR. Total RNA was prepared using Trizol reagent (Invitrogene, Carlsbad, Calif.) from 15 of normal and defective embryos and treated with RNase-free DNase to remove genomic DNA contamination for 30 min at 37 C° prior to reverse transcription. Three •g of each total RNA were used for 1st strand cDNA synthesis in a final volume of 30 •l using superscript III (Invitrogen, Carlsbad, Calif.) according to manufacture's protocol. The primers used in the PCR were described in Table 4. Assessment of relative levels of the expression of hematopoietic marker gene was carried out and determined by relative quantitative real-time RT PCR as previously described¹⁵. Briefly, a series of 10-fold dilution of 1^{st} strand cDNA was used as a standard molecule and the 1^{st} strand cDNA synthesized from total RNA of normal and defective embryos was amplified parallel to the standard molecule. Amplification primers for each specific mRNA are described in Table 6. PCR conditions are as follows: denaturation at 95° C. for 3 min and followed by 50 cycles of amplification (95° C. for 20 sec, 60° C. for 15 sec, 72° C. for 20 sec). A melting curve program (95-40° C. with a heating rate of 0.1° C./s) was also included to confirm the specificity of the amplification. PCR efficiencies of all reactions were between 95% and 100%. All measurements were performed in triplicate and repeated at 3 times. The data were analyzed using iCycler Thermal Cycler analysis software (Optical System Interface version 2.3).

TABLE 4
Primers used in conventional and real-time RT-PCR

| Primer | | |
|---|---|---|
| No. | Sequence (5′ → 3′) | Target gene |
| TTC882 | GGTCCAGTAGCCCTTTCC | GATA1 cDNA (sense) |
| TTC883 | ACTGTCTTTCCCATCACG | GATA1 cDNA |
| | | (antisense) |
| TTC879 | GCCACACCTCATACACAG | GATA2 cDNA (sense) |
| TTC880 | AGGAAACTGGAGCCGTGC | GATA2 cDNA |
| | | (antisense) |
| TTC1008 | GTCCTTCTCTCACATCTC | Fli1a cDNA (sense) |
| TTC1010 | CTCTCCGTTGGTTCCTTC | Fli1a cDNA |
| | | (antisense) |
| TTC1012 | GAACTACAATCACACACC | c-myb cDNA (sense) |
| TTC1013 | GTAGTGTCTCTGGATAGC | c-myb cDNA |
| | | (antisense) |
| TTC1016 | ATCAATGCCACAAACCTG | Mpx cDNA (sense) |
| TTC1017 | GGTTCTTCCGATTGTTGC | Mpx cDNA |
| | | (antisense) |
| TTC1020 | GCGGTGGGAGACGGCATC | L-plastin cDNA |
| | | (sense) |
| TTC1021 | TTCAAGTTCTCCTGTATG | L-plastin cDNA |
| | | (antisense) |
| TTC1024 | AACCTGCTCCGACACATC | Ikaros cDNA (sense) |
| TTC1025 | CTGCTTGTAACTGCGTCC | Ikaros cDNA |
| | | (antisense) |
| TTC830 | ATCTGGCATCACACCTTC | β-actin (sense) |
| TTC831 | CCATCACCAGAGTCCATC | β-actin (anti- |
| | | sense) |

TABLE 5
Phenocopy of defective embryos in zebrafish
embryos microinjected with rtEa4-peptide

| Microinjected | # of injected | # of survived | # of defective |
|---|---|---|---|
| peptide* | embryos | embryos | embryos |
| rtEa4-peptide | 624 | 574 | 370 |
| rtEa3-peptide | 400 | 348 | 0 |
| BSA** | 400 | 370 | 0 |

*One pmol of each peptide was injected into embryos at 2.5 hpf.
**BSA, bovine serum albumin

Diaminofluorene (DAF) staining for hemoglobin in zebrafish embryos. 2,7-diaminofluorene (DAF) stain has been used to detect terminal erythropoietic differentiation in erythroid cell. The hemoglobin-induced sensitive pseudo-peroxidase oxidation of DAF leaves cells containing hemoglobin with a deep blue coloration easily detectable on low magnification in a quantitative manner. Developing zebrafish embryos at 28 hpf and 35 hpf were examined under dissecting microscope after DAF staining for 20 min (FIGS. 3A and B). A deep blue coloration of cells indicates hemoglobinized red blood cells in yolk sac and body of the embryos. The first defection of the blue stain of hemoglobinized red blood cell was at 20-21 hpf in zebrafish embryonic development (data not shown). Circulation of the deep blue stained red blood cells was captured in live-image at 30 and 45 hpf (see movie clips in supplementary data). This result suggests that DAF staining is an effective method for the detection of mature erythrocyte in fish embryonic development. Furthermore, fish embryos survived after DAF staining if they are cultured in embryo medium in absence of DAF stain solution.

Microinjection of recombinant rtEa4-peptide into developing zebrafish embryos at 2.5 hpf. Injection of 1 pmole, recombinant rtEa4-peptide into embryos at 2.5 hpf resulted in the reduction of red blood cells accompany with defects in heart and vasculature development as previously reported. As negative controls, synthetic rtEa3-peptide and bovine serum albumin (BSA) were microinjected. Out of 574 survived embryos, 370 embryos showed developmental defects in heart, red blood cell, and vasculature. In embryos received both negative control proteins, no defective embryo was found (Table 5).

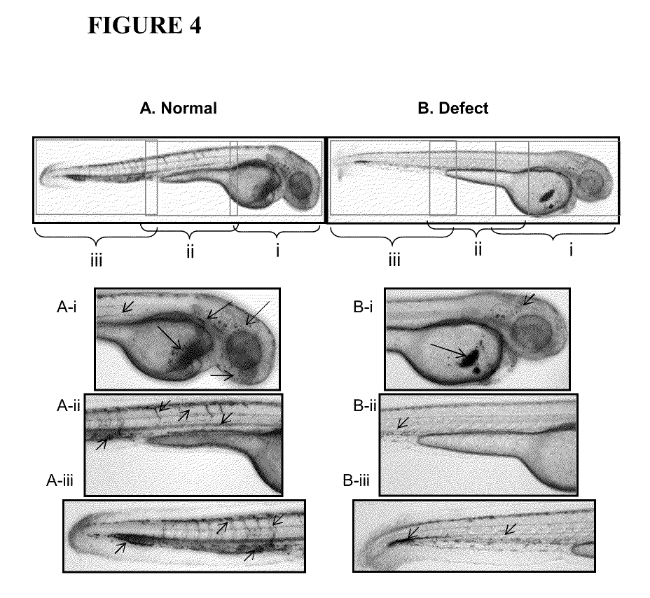

Reduced hemoglobinized red blood cells were captured by diaminofluorene staining in rtEa4-received embryos. Defective and normal embryos at 48 hpf were dechorinated and subjected to DAF staining for hemoglobin analysis as described in materials & methods. In FIG. 4A, hemoglobinized red blood cells in normal embryo at 48 hpf were shown in head, heart, yolk sac, body, and tail in the deep blue coloration. In contrast, the significant reduction of hemoglobinized red blood cells was shown in head, heart, yolk sac, body, and tail defective embryos (FIG. 4B).

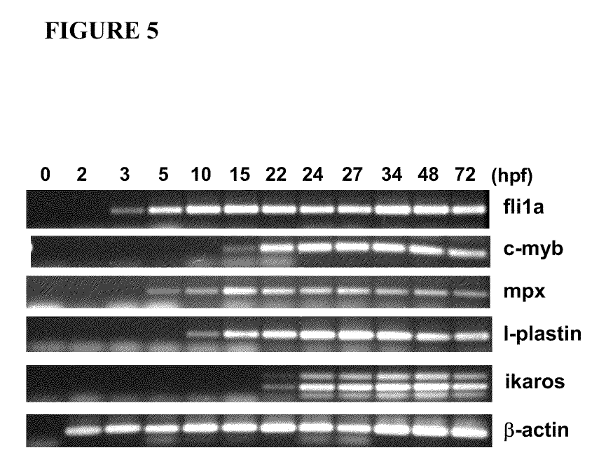

Temporal expression of hematopoietic marker genes, fli1a, c-myb, mpx, l-plastin, and ikaros in embryonic development. The temporal expression patterns of fli1a, c-myb, mpx, l-plastin and ikaros genes were determined by RT-PCR during embryonic development of normal embryos. As shown in FIG. 5, the mRNA of fli1a gene was first detected at 3 hpf, c-myb at 15 hpf, mpx at 5 hpf, l-plastin at 10 hpf, and ikaros at 22 hpf. Since intermediate cell mass (ICM) appears at 18 hpf, circulation begins at 24 hpf and the indication of lymphopoiesis in thymus appears at 65 hpf in zebrafish, the onset of expression of fli1a, c-myb, mpx, l-plastin and ikaros genes occurs much earlier than these events.

The mRNA level of gata1, gata2, fli1a, c-myb, mpx, l-plastin, and ikaros is determined in normal and defective embryos by relative quantification using real-time RT-PCR. Since the introduction of recombinant rtEa4-peptide into zebrafish embryos at 2.5 hpf resulted in developmental defects in erythropoiesis, we further analyzed the expression of hematopoietic marker genes related to primitive progenitor cell (fli1a and gata2), definitive hematopoietic stem cell (c-myb), neutrophil (mpx), momocyte (l-plastin), erythrocyte (gata1) and lymphocyte (ikaros) in defective embryos. The mRNA level of gata1, gata2,fli1a, c-myb, mpx, l-plastin, and ikaros gene in normal and defective embryos at 36 hpf, were determined by relative quantification using real-time RT-PCR analysis. As shown in FIG. 5, the mRNA level of gata1, fli1a, c-myb, mpx, and l-plastin genes are significantly reduced in range of 11.3 to 36.6-fold in defective embryos. However, the mRNA level of gata2 and ikaros genes are slightly reduced in range of 1.8 to 2.4-fold, respectively.

TABLE 6
Fold of reduction of expression of selective
hematopoietic genes in zebrafish embryos injected
with rtEa4-peptide (1 pmol) at 2.5 hpf.

| Hematopoietic Marker Genes | *Fold of Reduction |
|---|---|
| gata1 | 12.3 ± 1.5 |
| gata 2 | 1.8 ± 1.1 |
| fli 1a | 36.6 ± 1.1 |
| c-myb | 11.3 ± 1.0 |
| mpx | 26.3 ± 1.1 |
| l-plastin | 17.4 ± 1.8 |
| Ikaros | 2.4 ± 1.2 |
| β-actin | 1.5 ± 1.2 |

*Fold of reduction = ½^{-(Δ} [symbol] CT), where Δ [symbol] CT is Δ CT sample − ΔCT control

Overexpression of rtEa4- or hEb-transgene in embryos of medaka and zebrafish resulted in developmental defects in heart, red blood cells, and vasculature. In defective embryos carrying rtEa4- or hEb-transgene, the expression levels of genes related to cardiogenesis (GATA5 and NKX2.5), erythropoiesis (GATA1 and GATA2), and vasculogenesis (VEGF) were shown to be reduced significantly. Furthermore, microinjection of recombinant rtEa4-peptide at 2.5 hpf disrupted early development in heart, red blood cells, and vasculature in dose-dependant manner.

In current studies we further investigated the defective hematopoiesis in embryos that were received recombinant rtEa4-peptide at 2.5 hpf. DAF staining was adapted from in vitro studies to analyze defective development of hemoglobin in developing embryos. While the benzidene family of stains are both toxic and may stain leukocytes, 2,7-diaminofluorene (DAF) stain leaves cells containing hemoglobin with a deep blue coloration through the hemoglobin-induced sensitive pseudo-hemoglobin oxidation of DAF. After DAF staining for 20 min, embryos were cultured in embryo buffer in absence of DAF staining solution and about 90% of embryos were survived to hatching (Data not shown). Furthermore, we were able to produce live-images of circulating red blood cells in a deep blue stain (see movie clips in supplementary data). Screening of defective hematopoietic development of zebrafish embryos using DAF staining was shown to be very effective, since dramatic reduction of a deep blue coloration of the cells was observed in defective embryos (FIG. 4). In zebrafish, embryonic hematopoiesis occurs at the intermediate cell mass (ICM) (which is further separated into anterior and posterior regions located in the truck ventral to the notochord, and the rostral blood island (RBI) arising from the cephalic mesoderm. Fewer amounts of red blood cells in RBI and posterior ICM and a trace of blue stain in anterior ICM of defective embryos were detected by DAF staining (FIG. 4B).

Temporal expression pattern of hematopoietic marker genes in normal embryonic development showed that mRNA of fli1a, c-myb, mpx, l-plastin, and ikaros genes appears at 3 hpf, 15 hpf, 5 hpf, 10 hpf, and 22 hpf and expressed relatively steady from 34 hpf to 72 hpf. The expression level of mRNAs of gata1, gata2, fli1a, c-myb, mpx, l-plastin and ikaros genes was determined by relative quantification using real-time RT-PCR in both normal and defective embryos at 36 hpf. The mRNA level of fli1a and gata2, marker genes for primitive progenitor cell, was reduced as 36.6 and 1.8-fold in defective embryos compared to that of normal embryos.

The mRNA level of c-myb, mpx, l-plastin, gata1 and ikaros genes, which are marker genes for definitive HSC, neutrophil, monocyte/macrophage, erythrocyte and lymphocyte, are also reduced as 11.3, 26.3, 17.4, 12.3, and 2.4-fold in defective embryos. Although the mRNA level of fli1a, c-myb, mpx, l-plastin, and gata1 genes was significantly reduced in defective embryos, it is unclear whether rtEa4-peptide directly affect the expression of marker genes in primitive hematopoiesis and the consequence of that cause the reduction of the mRNA of genes related to definitive hematopoiesis, or rtEa4-peptide affects the expression of both primitive and definitive hematopoietic marker genes. In the previous studies we observed 3 or 2 different phenocopies of embryos carrying rtEa4- or hEb-peptide transgene in medaka and zebrafish, termed as group I, II, and III following arrest of heart development at cardiomyocyte, heart tube, and heart looping. However, microinjecting recombinant rtEa4-peptide into zebrafish embryos at 2.5 hpf resulted in only group I accompanying with defects on vasculogenesis and hematopoiesis in dose dependant manner. These results suggest that the pleotrophic effects of rtEa4-peptide in transgenic studies may due to the various temporal expressions of the transgene as well as the various amounts of the transgene product in embryonic development.

The zebrafish has developed as an ideal organism for the study of hematopoiesis and other aspects of embryogenesis and organogenesis. The externally fertilized embryos are optically clear and easily maintained and manipulated. Several large-scale forward genetic screenings have generated thousands of mutants in the past decade. Recently, our laboratory has reported an effective reverse genetic approach for the study of cardiogenesis, red blood cell and vasculature development by introducing E-peptides, for example, rtEa4-peptide into developing fish embryos. Diaminofluorene (DAF) staining is an effective method for staining hemoglobinized red blood cells in developing zebrafish embryos. We applied the DAF stain to analyze the defective erythropoiesis in zebrafish embryos that received rtEa4-peptide. The mRNA level of hematopoietic marker genes, gata1, fli1a, c-myb, mpx, and l-plastin were examined by relative quantification using real-time RT-PCR in normal and defective embryos and shown to be significantly reduced ranging in 11.3 to 36.6-fold in defective embryos.

Introduction of a transgene encoding rtEa4- or hEb-peptide into newly fertilized medaka or zebrafish embryos resulted in disruption of development in heart, red blood cell, and vasculature. See, Chun and Chen, Comp. Biochem. Physiol., Part C, 145:39-44 (2007), incorporated herein by reference. In medaka, overexpression of rtEa4- or hEb-peptide caused three different arrests of heart development at cardiomyocyte, heart tube, and heart looping stages termed as group I, II, and III. In zebrafish, we observed defective group I and II in embryos microinjected rtEa4- or hEb-peptide transgene. In all defective embryos of medaka and zebrafish, significant reduction of red blood cell and vasculature development was accompanied with defective heart development.

Furthermore, mRNA level of genes related to development of heart (gata5 and nkx2.5), red blood cell (gata1 and gata2), and vasculature (vegf) was significantly reduced in defective embryos. Although molecular mechanism of disruption of heart, red blood cell, and vasculature development remains elusive, these results suggested that the inhibitory effect of rtEa4-peptide on heart, red blood cell and vasculature development could be the consequence of down regulation of the expression of gata5, nkx2.5, gata1, gata2, and vegf genes. Microinjection of recombinant rtEa4-peptide into developing zebrafish embryos at 2.5 hpf resulted in perturbation of development in heart, red blood cells, and vasculature similar to group I in transgenic studies and the disruption of development in heart, red blood cell, and vasculature by recombinant rtEa4-peptide showed dose-dependant manner.

Primitive or embryonic hematopoiesis predominantly produces erythrocytes, as well as some primitive macrophages. In mammals, the primitive hematopoiesis is found in the extraembryonic yolk sac where early erythrocytes are generated. In zebrafish, primitive hematopoiesis occurs in two intraembryonic locations: the intermediate cell mass (ICM) located in the truck ventral to the notochord, and the rostral blood island (RBI) arising from the cephalic mesoderm. Within the posterior mesoderm, cells lateral to the developing somites express both vascular and blood markers and migrate medially around 18 hpf to fuse at the midline forming the ICM. Cells within the ICM, equivalent to the mammalian yolk sac blood island, differentiate into the endothelial cells of the trunk vasculature and proerythroblast, which begin to enter the circulation around 24 hpf. Cells in the anterior mesoderm of the zebrafish embryo make up a second anatomical site for hematopoiesis, known as RBI, which predominantly generates macrophage. Two hematopoietic marker genes, draculin and leucocyte-specific plastin (l-plastin), are expressed in macrophages which appear in the embryo as early as erythroid cells. GATA1, a zinc finger transcription factor, is essential for primitive erythropoiesis. Gata1 transcripts are expressed bilaterally in the lateral plate mesoderm that will migrate medially to form the ICM. Transgenic zebrafish carrying the gata1 promoter driving expression of green fluorescent protein showed that the gata1+ cells in the ICM differentiate into proerythroblasts and enter the circulation around 24 hpf. Like the primitive progenitors in the ICM, those found in the RBI contribute to both blood and vascular development, and express several transcription factors, including fli1a, gata2, lmo2, and scl. The cells seen in the RBI are morphologically identifiable as macrophages, and are first noted in the lateral head mesoderm around 11 hpf. Myeloperoxidase (mpx/mpo), an enzyme that is a major component of neutrophil and eosinophil granules, is a marker for zebrafish granulocytes and some early mpo+ cells are found over the anterior yolk sac. The function of early macrophages has been observed as early as 26 hpf in the ducts of Cuvier, where macrophages can observed engulfing apoptotic erythroid cells.

The present invention confirms that E-peptides, for example, rtEa-4-peptide and hEb-peptide, have novel biological activities. These activities include the inhibition of hematopoiesis; i.e., the differentiation of blood cell progenitors. This observation is both surprising and unexpected because it has never been demonstrated that E-peptides can delay differentiation of stem cells.

Although the biological function(s) of E-peptide in embryonic development remains to be elusive, our results suggest that rtEa4-peptide could be used in reverse genetics for dissecting genes involved in hematopoiesis in lower vertebrates.

### EXAMPLE 15

### Differentiation of Neuronal Progenitor Cells

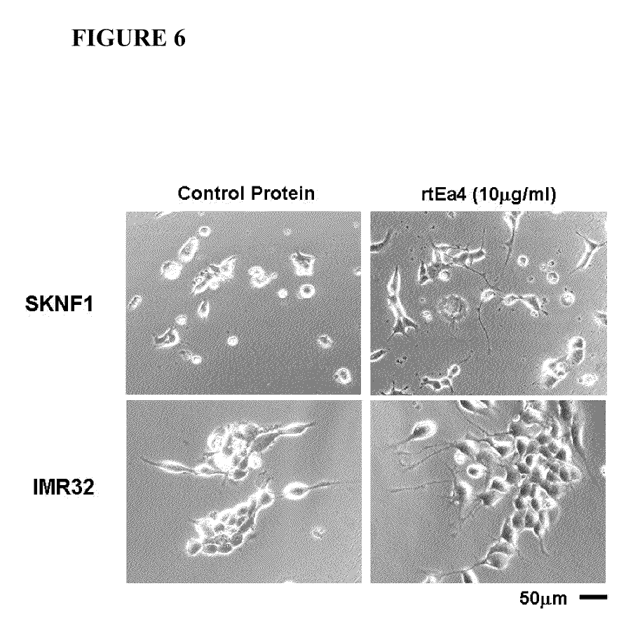

FIG. 6 shows the effect of trout Ea4-peptide of pro-IGF-I on induction of morphological differentiation of human neuroblastoma cells (SK-N-F1 and IMR32). SK-N-F1 and IMR32 cells are cultured in a DMEM/F12 (1:1) medium supplemented with 10% FBS and 10 μg/ml trout Ea4-peptide. Twenty hours after the addition of trout Ea4-peptide, the cells are observed and photographed under a microscope (IX50, Olympus). Results of the study showed that trout Ea4-peptide induced morphological differentiation of SK-N-F1 and IMR32 cells.

Table 7 shows the effect of rtEa4- or hEb-peptide on up-regulation of expression NPY and PTEN, and down-regulation of c-Jun and N-Myc genes in SK-N-F1 and IMR 32 cells. It has been shown by other investigators that increased expression of NPY and GAP-43 genes was observed human neuroblastoma cells during differentiation (Anderson et al., (1994). Furthermore, decreased expression of N-Myc and c-Jun genes and increased expression of PTNE gene have also been observed in neuroblastoma cells going through differentiation. Therefore, one would expect to detect increased expression of NPY and PTEN genes and decreased expression of N-Myc and c-Jun genes in E-peptide-induced morphological differentiation of neuroblastoma cells. To prove this hypothesis, total RNA samples were extracted from SK-N-F1 and IMR 32 cells with and without treatment with 2 μg/ml for 6 hours by the guanidinium thiocyanate-phenol-chloroform method. The levels of NPY, c-Jun, N-Myc and PTEN mRNA were determined by quantitative real-time RT-PCR method and data expressed as: Relative mRNA level=2^{-[S•CT-C•CT]}. Each data point was the average of at least 6 determinations.

As shown in Table 2, while levels of NPY, and PTEN mRNA in E-peptide treated cells were substantially increased, the levels of N-Myc nd c-Jun mRNA were highly reduced by E-peptide treatment. These results support the observation that E-peptide induces the differentiation of SK-N-F1 IMR 32 cells.

TABLE 7
Relative expression levels of NPY,
N-Myc, c-Jun, and PTEN genes.

Relative Level of Expression

| Gene | SK-N-F1 | IMR 32 |
|---|---|---|
| NPY | 2.06 ± 0.67 | 7.14 ± 0.45 |
| N-Myc | 0.45 ± 0.01 | 0.66 ± 0.03 |
| C-Jun | 0.37 ± 0.03 | 2.06 0.04 |
| PTEN | 11.11 ± 2.54 | 3.42 ± 0.27 |

Relative Level of Exression = 2 − (Δ [symbol] CT), where Δ [symbol] CT is Δ CT sample − ΔCT control.
Relative level of expression > 1, up-regulation; <1, down regulation

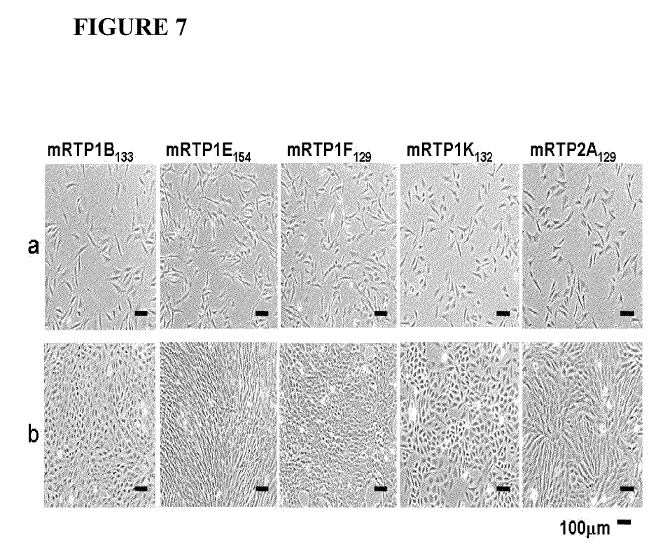

FIGS. 7-8 show the effect of rtEa4- or hEb-peptide on establishing permanent pituitary cell lines from primary cells. It is generally believed that endocrine cells are differentiated from neuronal stem cells. In an attempt to develop permanent pituitary cell lines from primary pituitary cells, we experienced tremendous difficulties. Primary trout pituitary cells cultured in a CO₂-independent medium supplemented with 10% FBS and 5 ng/ml of bFGH died out in less 14 days. However, by culturing the same trout primary pituitary cells in the same medium supplemented with rtEa4-or hEb-peptide (4 μg/ml), these cells continue to grow passed 120 passages and became permanent cell lines. These cells maintain the characteristics of synthesizing GH and PRL as shown by immunocytochemical analysis. These results further support the notion that E-peptide can be used to propagate and differentiate neuronal stem cells.

While this invention has been particularly shown and described with references to exemplary and preferred embodiments thereof, it will be understood by those skilled in the art that various changes in form and details may be made therein without departing from the scope of the present invention encompassed by the appended claims.

1. A method for inhibiting the differentiation of a progenitor cell, comprising treating the progenitor cell with a composition comprising an effective amount of an insulin-like growth factor (IGF-1) E-domain peptide together with at least one of a pharmaceutically acceptable carrier, excipient or adjuvant, wherein the composition inhibits cell differentiation.

2. The method of claim 1, wherein the progenitor cell is a stem cell.

3. The method of claim 1, wherein the progenitor cell is a hematopoietic stem cell.

4. The method of claim 3, wherein the neuronal stem cell is a pituitary stem cell.

5. The method of claim 1, wherein the composition comprises at least one E-domain peptide having the amino acid sequence as set forth in SEQ ID NO:2, having the amino acid sequence as set forth in SEQ ID NO:1, or a combination of both.

6. The method of claim 5, wherein the composition comprises a fusion protein having an E-domain peptide joined in a contiguous polypeptide chain with a non-E-domain peptide.

7. A cell generated according to the method of claim 1.

8. A method for promoting the differentiation of a neuronal progenitor cell, comprising treating a neuronal progenitor cell with a composition comprising an effective amount of an E-domain peptide together with at least one of a pharmaceutically acceptable carrier, excipient or adjuvant, wherein the composition promotes differentiation of the neuronal progenitor cell.

9. The method of claim 8, wherein the neuronal progenitor cell is at least one of a primary neuronal stem cell or a neuronal tumor progenitor cell.

10. The method of claim 9, wherein the primary neuronal stem cell is a pituitary stem cell.

11. The method of claim 9, wherein the neuronal tumor progenitor cell is a neuroblastoma cell.

12. The method of claim 8, wherein the composition comprises at least one of an E-domain peptide having the amino acid sequence as set forth in SEQ ID NO:2, having the amino acid sequence as set forth in SEQ ID NO: 1, or a combination or both.

13. The method of claim 12, wherein the composition comprises a fusion protein having an E-domain peptide joined in a contiguous polypeptide chain with a non-E-domain peptide.

14. A method for the suppression of the malignant growth of a neuroblastoma cell comprising, treating a neuroblastoma cell with an effective amount of an E-domain peptide together with at least one of a pharmaceutically acceptable carrier, excipient or adjuvant, wherein the composition promotes neuroblastoma cell differentiation.

15. The method of claim 14, wherein the composition comprises at least one of an E-peptide having the amino acid sequence as set forth in SEQ ID NO:2, having the amino acid sequence as set forth in SEQ ID NO:1, or a combination of both.

16. The method of claim 15, wherein the composition comprises a fusion protein having an E-domain peptide joined in a contiguous polypeptide chain with a non-E-domain peptide.
